(19) United States
(12) Patent Application Publication
(10) Pub. No.: US 2005/0287611 A1
(43) Pub. Date: Dec. 29, 2005

(54) MHC bridging system for detecting CTL-mediated lysis of antigen presenting cells

(76) Inventors: C. Thomas Nugent IV, San Diego, CA (US); Abhay Kumar, San Diego, CA (US); Kristine Kuus-Reichel, San Diego, CA (US)

Correspondence Address: BECKMAN COULTER, INC.;C/O DLA PIPER RUDNICK GRAY CARY US LLP, 4365 EXECUTIVE DR, SUITE 1100, SAN DIEGO, CA, 92121-2133, US

(21) Appl. No.: 11/123,829

(22) Filed: May 5, 2005

Related U.S. Application Data
(60) Provisional application No. 60/568,900, filed on May 7, 2004.

(51) Int. Cl. G01N 33/574

(52) U.S. Cl. 435/7.23

(57) ABSTRACT

A bridging assay that utilizes a multivalent MHC binding molecule to enumerate the number of antigen-specific CTLs in a particular sample and also determines the functional capability of the CTL population in the sample is provided. In one embodiment, the assay is used to measure the effector function of any tetramer-positive CTL using a single non-MHC-containing target cell line that is adapted to form an antibody bridge with the tetramer. Furthermore, effector function and enumeration can be measured by flow cytometry, and additional markers residing on either effector or target cell populations may be detected using antibodies coupled with other fluorochromes. The tetramer bridging assay will allow investigators to easily determine the lytic capacity and antigenic specificity of CTLs using a commercially available reagent in a non-radioactive assay.

### CROSS REFERENCE TO RELATED APPLICATION(S)

This application claims the benefit of priority under 35 U.S.C. § 119(e) of U.S. Ser. No. 60/568,900, filed May 7, 2004, the entire content of which is incorporated herein by reference.

### BACKGROUND OF THE INVENTION

This invention is related to immunoassays and, in particular, to assays for detecting CTL-mediated lysis of antigen presenting cells.

Cytotoxic or cytolytic T lymphocytes (CTLs) are a component of the adaptive immune response and are responsible for destroying cells that are infected with viruses or other intracellular pathogens. In order to perform this function, CTLs must be able to delineate self-antigens from nonself antigens. Mature CTLs that have successfully undergone thymic selection possess a polymorphic T cell antigen receptor (TCR) capable of binding class I MHC. Class I MHC is ubiquitously expressed on the majority of nucleated cell surfaces throughout the host, and possesses a binding pocket for a peptide derived from a degraded intracellular protein, either self or pathogenic in origin. Recognition of a foreign peptide/MHC complex will trigger activation of CTL through the TCR and ultimately cause destruction of the target cell displaying the TCR. Activated CTLs can destroy a target cell through a variety of mechanisms, including secretion of cytokines, the Fas receptor (CD95/CD95L) pathway, and exocytosis of cytolytic granules. The latter mechanism is the primary mode of contact-mediated cytotoxicity (D. Kagi et al., Annu Rev Immunol 1996; 14:207-32). In theory, all mature CTLs are capable of lysing target cells. However, infection with some viruses can modulate effector functions of CTLs, especially those that are antigen-specific for the pathogen, such as HCV or HIV.

Since the magnitude of an immune response can assist clinicians in following the progression and determining the prognosis of an infection, a great deal of effort has gone into developing methods for measuring the level and persistence of an immune response. For a humoral response, simple methods are available for determining the circulating levels of antibodies in the serum. Methods for measuring the magnitude of a cellular immune response, however, are not as straightforward, since they generally require identifying the T cells involved in the response.

A common method for determining the number of T cells in an individual that are responsive for a particular antigen is the limiting dilution assay (LDA). In this method, CTLs are serially diluted in microtiter plates until a single cell on average is present in a well, then the cells are stimulated to proliferate, and examined for cytotoxic activity in response to antigen. This method is useful because it indicates not only that the CTLs have cytotoxic activity, but also that the CTLs can proliferate, which can be critical upon subsequent infection. Unfortunately, the limiting dilution assay is time consuming because the CTLs generally need to proliferate for a couple of weeks to produce a sufficient number to measure cytotoxic activity. Thus, the assay is labor intensive and expensive to perform, and is not readily adaptable to a high throughput assay format. In addition, the limiting dilution assay may underestimate the number of specific CTLs in an individual because the method only identifies CTLs that have the capacity to proliferate.

Another method that has been useful for identifying antigen-specific CTLs relies on the expression of cytokines such as interferon gamma by antigen stimulated CTLs. In this method, antigen stimulated cells are permeabilized, and intracellular immunostaining is performed using, for example, detectably labeled anti-interferon gamma antibodies. This method has advantages over the limiting dilution assay because there is no requirement for cell proliferation or, therefore, a cell-culturing step, and it can be readily adapted to a high throughput assay format. However, the method is toxic to the cells and, therefore, it is not possible to select live antigen-specific cells, for example, to perform additional functional tests.

A more recently developed method of detecting antigen-specific T cells utilizing tetramers of major histocompatibility complex (MHC) molecules has revolutionized T cell analysis. MHC tetramer complexes are formed by the association of four MHC monomers, for example, four MHC class I molecule/β2-microglobulin monomers, with a specific peptide antigen and a detectable label such as a fluorochrome held together by a multivalent entity, such as streptavidin. Such MHC class I molecule tetramer complexes bind to a distinct set of T cell receptors on a subset of CD8+ T cells, including cytotoxic T lymphocytes (CTLs). CTLs, which are effector CD8+ T cells, do not necessarily represent the whole antigen-specific pool of CD8+ T cells. In this respect, the LDA and cytokine assay both detect CTLs or subpopulations of CTLs; whereas the MHC tetramer method can detect all antigen-specific CD8+ T cells, including naive and anergic CD8+ T cells, which do not exhibit effector functions. Mixing the MHC tetramers with peripheral blood lymphocytes or whole blood, and using flow cytometry as a detection system provide a count of all T cells that are specific for a peptide and its matched allele. Thus, the MHC tetramers allow for measurement of a cellular response against a specific peptide.

The use of MHC tetramers to analyze T cell specificity provides significant advantages over previously used T cell assays. For example, the MHC tetramer method is quantitative; it does not require the use of radioactive labels; and it is readily adapted to high throughput assay formats. In addition, the method can be performed quickly and, therefore, can be used to examine fresh blood or tissue samples. Where the MHC tetramer complex includes a fluorescent label, a cell population including T cells can be further stained with one or more other fluorescently labeled molecules, for example, fluorescently labeled molecules specific for other cell surface molecules and analyzed using flow cytometry, thus allowing additional characterization of the responding cells. In this case, the additional fluorescent label is selected to fluoresce at a wavelength that is readily distinguishable from the label(s) used to stain the target cells. Furthermore, MHC tetramer analysis is not toxic to the labeled cells and, therefore, tetramer binding cells can be sorted into uniform populations by flow cytometry and examined by additional assays to confirm their functional ability, for example, the ability to proliferate in response to antigen.

The use of MHC tetramer analysis allows identification of individual T cells on the basis of the specificity of their binding to the MHC-peptide complex. The tetramer analysis method has been used to study CD8+ T cell responses in humans with acute viral infections such as HIV, where it revealed that the increase of antigen-specific CD8+ T cells during the acute phase of the response was far greater than previously thought. MHC tetramers also have been used to accurately and efficiently monitor CD8+ T cell responses in other viral infections, including Epstein Barr virus-mononucleosis, cytomegalovirus, human papilloma virus, hepatitis B, hepatitis C, influenza and measles; in a parasitic infection, malaria; in cancers, including breast, prostate, melanoma, colon, lung, and cervical cancers; in autoimmune diseases, including multiple sclerosis and rheumatoid arthritis; and transplantation.

However the MHC tetramer assay does not provide information regarding the ability of the MHC monomer to activate antigen-specific CTLs to lyse target cells (i.e. to have “effector function). Historically, investigators have been primarily interested in not only measuring the lytic potential of antigen-specific CTLs, but also redirecting effector function to a cell line that does not express the MHC/peptide ligand. The concept of redirecting CTL effector function was originally reported in the mid 1980s. Investigators constructed chemically crosslinked antibodies, called heteroaggregates, heteroantibody duplexes or hybrid antibodies. These complexes were comprised of an antibody specific for TCR or CD3 and an antibody specific for a unique cell surface protein expressed by the target cell. Target cells either expressed the protein endogenously or after decoration with a hapten, such as dinitrophenol. Later reports documented that this method was not restricted solely to CTL clones, and could be used to redirect the effector function of fresh human peripheral blood mononuclear cells (PBMC) (Jung G., et al., Proc Natl Acad Sci USA 1986 June; 83(12):4479-83, Perez, supra). Other laboratories constructed polystyrene beads that expressed both receptor-triggering antibody (i.e., anti-CD3) and antibody (Ab) specific for a target cell surface protein. All of these methods involved the chemical cross linking of Ab fragments, or fusion of two hybridomas (called “quadromas”) to create a cell line that would secrete antibody mixtures that included molecules with specificity for both the TCR and the tumor antigen. At the time, these types of approaches yielded heterologous mixtures of antibodies, and a homogenous solution was difficult to obtain.

Later, a recombinant approach was taken. Functional single-chain bispecific Ab (scFv₂) were engineered for expression in E. coli. The recombinant construct contained the V_{H} and V_{L} genes from an anti-TCR Ab, and the V_{H} and V_{L} genes from an anti-fluorescein Ab. The resulting construct was used to redirect CTL-mediated lysis to fluorescein-decorated tumor cells. Other laboratories used a eukaryotic expression/secretion system to produce bispecific antibody, but the concept of bridging a CTL to a target by antibody specific for the TCR on one end, and the target cell on the other remained the same. However in all of these approaches mentioned so far, redirected lysis of CTL involved coupling of the CTL to the target in a fashion that was not TCR-specific. CTL-binding portions of the bridging molecules could bind all TCR, or the non-polymorphic CD3 portion of the TCR supramolecular complex. The antigenic specificity of the CTL was never shown.

Robert et al. (Eur J Immunol 2000 November; 30(11):3165-70) reported an alternative approach to redirecting CTL lysis. In this system, redirection of the lysis of CTLs with known lytic capability can be accomplished. Antigen-specific CTLs were associated with target cells by Fab′ specific for tumor antigens chemically coupled to tetramers, or complexes of biotinylated MHC and peptide bound together by streptavidin, which naturally has 4 biotin-binding sites. (FIG. 1). Target cells were first coated with the Fab′-modified tetramers, and then incubated with an antigen-specific CTL line or clone of interest. Therefore, in this approach, the conjugate was selective for CTLs with a particular antigenic specificity, and tumor cell lines were decorated to express the particular antigen/MHC complex. Studies from other laboratories have focused on exploiting the binding properties of streptavidin, or genetically modifying the streptavidin itself. Ogg et al. (Br J Cancer 2000 March; 82(5):1058-62) linked tumor antigen-specific antibodies and tetramers loaded with single chain MHC by biotinylating both molecules so as to bind streptavidin. Later studies from this laboratory used a recombinant fusion protein comprised of wild-type streptavidin and anti-CD20 Ab. This fusion protein possessed four binding sites for an antigen expressed on the surface of a target cell, in addition to retaining all four biotin-binding sites. Biotinylated MHC/peptide monomers were coupled to the fusion protein and used to successfully redirect the effector function of PBMCs that had undergone an initial round of stimulation.

Other investigators have reported that CD8+ T lymphocyte populations isolated from individuals undergoing an immune response to HCV and HIV can bind tetramer in a disease and antigen-specific fashion. However, these populations were determined to be “stunned” or anergic, as not all tetramer-positive lymphocytes possessed the capability to secrete the lymphokine IFN (Lechner, R, et al., J. Exp. Med. (2000) 191:1499-1512). However, the presence or absence of CTLs within a lymphocyte culture has classically been defined by the presence or absence of effector function. CTLs, by definition, are able to lyse a target cell in an antigen-specific fashion, by secreting lytic granules that contain perforin and granzymes.

The presence of CD8⁺ T lymphocytes (CTL) in vitro is routinely measured by their functional capacity to lyse antigen-presenting target cells. This function can be quantitated by radioactive and nonradioactive lytic assays that measure the overall effector function of the sample of interest. Enumeration of the CTLs responsible for the effector function and simultaneous detection of the effector function by these lymphocytes is technically challenging, and to date no technique has been reported that will accomplish this task.

Thus, in view of the above, new and better assays developed to determine CTL antigenic specificity and lytic capacity are needed to determine the role of CTLs in various diseases, and to quantitated effector function of antigen-specific CTLs. The present invention satisfies this need and provides additional advantages.

### SUMMARY OF THE INVENTION

The invention is based on the discovery that a tetramer-based bridging system connecting an artificial antigen-presenting cell with an antigen-specific cytotoxic T cell can be used to quantitate by radioactive and non-radioactive lytic assays the effector function of an antigen-restricted CTL or the overall effector function of a sample of interest.

Accordingly, in one embodiment the invention provides an assay for identifying a peptide of a known antigen that induces peptide-restricted effector function in a CTL by co-incubating under suitable conditions so as to allow interaction between the following four components: 1) a target cell with a surface ligand and a first detectable label, which produces a signal that is substantially changed in lysed target cells as compared to unlysed target cells; 2) a multimeric MHC monomer or modified MHC monomer complex with bound test MHC-binding peptide, wherein the complex further comprises an antibody-specific binding site; 3) an antibody that binds to the ligand on the target cell and to the antibody-specific binding site; and 4) a peptide-restricted CTL. The interaction between these components results in formation of a bridging complex that brings the peptide-restricted CTL into cell-lysing proximity of the target cells. A change detected in signal produced by target cell in the bridging complex as compared with signal produced by uncomplexed target cells identifies the peptide in the MHC class I monomer as inducing peptide-restricted effector function in the CTL.

In another embodiment, the invention provides a bridging complex containing the following components: 1) a cytotoxic T cell (CTL) with an antigen-restricted T cell receptor (TCR); 2) a multimeric MHC monomer or modified MHC monomer complex with bound MHC-binding peptide of a known antigen that induces peptide-restricted effector function in a CTL and an antibody-specific binding site; 3) a target cell having a surface ligand; and 4) an antibody that binds to each of the antibody binding site and the ligand. Immunological binding of the TCR to the monomer and binding of the antibody to each of the ligand and the antibody binding site generates the bridging complex such that the target cell and the CTL are brought into cell-lysing proximity.

In yet another embodiment, the invention provides methods for detecting in a sample containing mixed CTLs the presence of a peptide-restricted CTL having effector function for a cell bearing an MHC monomer with bound MHC-binding peptide of a known antigen. Practice of the invention methods involves contacting together under suitable conditions so as to allow binding between: 1) a sample comprising a mixture of CTLs of unknown specificity; 2) target cells with a known surface ligand and a first detectable label, wherein signal of the first detectable label is substantially decreased in lysed cells as compared with unlysed cells; and 3) an MHC multimeric complex formed by attachment to a multivalent entity of an antibody specific for the known surface ligand and a plurality of MHC monomers or modified MHC monomers, each with a bound MHC-binding peptide of the known antigen. Detection of a decrease in signal produced by the first detectable label resulting from the binding as compared with signal produced therefrom prior to the binding indicates the presence in the sample of one or more peptide-restricted CTLs with effector function for a cell bearing an MHC monomer having a bound MHC-binding peptide of the known the antigen.

In yet another embodiment, the invention provides methods for detecting antigen specific effector function of a CTL by contacting together under suitable binding conditions so as to allow binding between 1) an FcR-bearing target cell that has an anti-fluorophore antibody specifically bound to the cell by Fc/FcR interaction, wherein the cell is labeled with a first detectable label with a signal that substantially decreases upon cell lysis; 2) a tetramer comprising streptavidin, a fluorophore attached to the streptavidin, and two to four ternary complexes of an MHC monomer or modified MHC monomer having bound thereto an MHC-binding peptide of a known antigen, wherein the monomers are biotinylated and bound to the streptavidin via biotin; and 3) at least one of the antigen-specific CTLs. Detection of a decrease in signal produced by the first detectable label resulting from the binding as compared with signal therefrom prior to the binding indicates that the CTL has antigen-specific effector function for an antigen-presenting cell that presents the MHC-binding peptide.

In still another embodiment, the invention provides methods for determining effector function of a cytotoxic T cell lymphocyte (CTL) for a cell presenting an MHC monomer or modified MHC monomer having a bound MHC-binding peptide, by co-incubating under suitable conditions so as to allow interaction between 1) an FcR-bearing target cell tagged with a first detectable label having a signal that changes upon lysing of the target cell; 2) a multimeric moiety comprising at least one complex of an MHC monomer or modified MHC monomer with a MHC-binding peptide and at least one Fc-containing antibody, wherein the at least one monomer and the at least one antibody are bound to a multivalent entity through specific binding pairs; and 3) a CTL having an antigen receptor (TCR) specific for the complex. The interaction allows formation of a bridging complex by binding of the Fc of the antibody to the FcR and binding of the TCR to the complex, thereby bringing the CTL into cell-lysing proximity of the target cell. Detection of a change in signal from the detectable label resulting from formation of the bridging complex as compared with signal produced therefrom in the absence of formation of the bridging complex indicates effector function of the CTL for a cell presenting the MHC monomer or modified MHC monomer and the MHC-binding peptide.

### BRIEF DESCRIPTION OF THE DRAWINGS

FIG. 1 is a schematic drawing showing a bridging system wherein an antigen-specific CTL is associated with a tumor antigen-expressing target cell by a Fab′ antibody fragment specific for a tumor antigen expressing target cell and by chemical coupling the antibody to a bridging tetramer, which is a complex of four biotinylated MHC/peptide complexes bound together by streptavidin (SA), which naturally has 4 biotin-binding sites.

FIG. 2 is a schematic diagram illustrating the invention methods for causing lysing of a target cell brought into association with a CTL with effector activity by means of a tetramer-bridging molecule. Target cells have fluorescent anti-phycoerythrin (PE) Fab bound to the cell surface through Fc/FcR interaction. PE is chemically bound to the streptavidin portion of the tetramer. The tetramer-coated CTL lyses the target cell in an antigen-specific fashion, as determined by capability of the CTL to bind the tetramer bridge.

FIG. 3 is schematic representation illustrating flow cytometric analysis for differentiating target cells from CTLs by tagging of target cells with two fluorescent dyes (PKH-26) and carboxy fluorescein diacetate, succinimidyl ester (CFSE), the latter of which is known to leak from lysed cells while the former does not. A gate (R1) is drawn on dye-positive cells to measure the production of fluorescence by the target cells (including PKH-26 and CFSE). Then, measurement is made of the number of cells containing only CFSE (M1) in order to quantitate the amount of leakable dye remaining in the target cells. By subtraction, the number of cells that were lysed by CTLs having effector function can be calculated.

FIGS. 4A and 4B are dot plots showing fluorescent staining of HA2FLU.3, a CD8+ CTL clone generated to recognize and bind influenza matrix peptide in the context of HLA-A*0201, with flu iTAg^{PE tetramer} (FIG. 4A) and an irrelevant A2/Gag iTAg tetramer (FIG. 4B)

FIGS. 5A-5D are a series of histogram overlays of CFSE dye-labeled, anti-PE Ab coated P815 target cells incubated with A2/flu-specific CTL. The stippled light tracing=A2/gag iTAg-reacted CTL; the dark solid line tracing=A2/flu iTag-reacted. As the Effector:Target ratio (E:T) increased, a significant decrease in CFSE-labeled targets was detected in the samples containing A2/Gag-reacted CTLs.

FIG. 6 is a graph showing % cell lysis obtained at E:T with the conditions described in FIGS. 5A-5D.

FIGS. 7A-D area series of dot plots showing specific staining of mixed CTL clones using the invention bridging system and methods. FIGS. 7A shows staining of B7 CMV.16 CTL clone, which is restricted to CMV pp65 peptide in the context of HLA-B*0701, when incubated with specific CMV pp65 tetramer (FIG. 7A, left) or with an MHC-matched irrelevant tetramer B7/gp41 (FIG. 7A, right). FIG. 7B shows staining of HA2FLU.5, a CTL clone that behaves similarly to sister clone HA2FLU.3 used in earlier experiments, and binds relevant tetramer A2/flu with high avidity (FIG. 7B, left). These two CTL clones were mixed in ratios beginning at 1:0 HA2FLU.5:B7CMV.16, and ending with the opposite ratio (0:1).

FIGS. 8A-8L are a series of graphs showing staining of mixtures of the clones of FIGS. 7A-7B HA2FLU.5:B7CMV.16 with E:T ratios of 1:0, 5:1, 3:1, 1:3, 1:5 and 0:1. Samples were mixed and separated into two groups. One group was stained with A2/Flu tetramer (FIGS. 8A, C, E, G, I, and K), and the other group was stained with the reciprocal B7/CMV tetramer (FIGS. 8B, D, F, H, J, and L).

FIG. 9 is a graph of the results obtained in the experiment of FIGS. 8A through 8L.

### DETAILED DESCRIPTION OF THE INVENTION

The present invention provides compositions, methods, and kits for detecting and quantitating effector function of antigen-specific CTLs and for redirecting effector function of such CTLs to target cells in a sample containing or suspected of containing one or more MHC monomers presenting a peptide of a known antigen. The invention is based on the discovery of a bridging complex that enables use of the same target cell (and in certain cases the same target cell-ligand-antibody combination) to test multiple different CTL-antigenic peptide combinations.

As used herein, the terms “MHC monomer” and “HLA monomer” refer to a class I MHC heavy chain that maintains the ability to assemble into a ternary complex with an appropriate MHC-binding or HLA-binding peptide and beta-2 microglobulin under conditions conducive to such assembly. As used herein, the terms “modified MHC monomer” and “modified HLA monomer” refer to class I monomers as described above, but which have been engineered to introduce modifications as described below. These terms also encompass functional fragments of the MHC monomer that maintain the ability to assemble into a ternary complex with an appropriate MHC-binding or HLA-binding peptide and beta-2 microglobulin under renaturing conditions and to dissociate under denaturing conditions. For example, a functional fragment can comprise only the α₁, α₂, α₃, domains, or only α₁, α₂ domains, of the class I heavy chain, i.e., the cell surface domains, that participate in formation of the ternary complex. In another embodiment, modified MHC monomers can be class I heavy chain molecules, or functional fragments thereof, contained in a fusion protein or “single chain” molecule and may further include an amino acid sequence functioning as a linker between cell surface domains of the monomer, a detectable marker or as a ligand to attach the molecule to a solid support that is coated with a second ligand with which the ligand in the fusion protein reacts. Moreover the terms “modified MHC monomer” and “modified HLA monomer” are intended to encompass chimera containing domains of class I heavy chain molecules from more than one species or from more than one class I subclass. For example, a chimera can be prepared by substitution of a mouse H-2Kb domain for one of the three alpha domains in a human HLA-A2 fragment. Such a molecule is conveniently expressed as a single chain with optional amino acid linkers between subunits or as a fusion protein as is known in the art.

Preparation of Monomers

The Class I MHC in humans is located on chromosome 6 and has three loci, HLA-, HLA-B, and HLA-C. The first two loci have a large number of alleles encoding alloantigens. These are found to consist of a 44 Kd heavy chain subunit and a 12 Kd beta₂-microglobulin subunit, which is common to all antigenic specificities. For example, soluble HLA-A2 can be purified after papain digestion of plasma membranes from the homozygous human lymphoblastoid cell line J-Y as described by Turner, M. J. et al., J. Biol. Chem. (1977) 252:7555-7567. Papain cleaves the 44 Kd heavy chain close to the transmembrane region, yielding a molecule comprised of α₁, α₂, α₃ domains and beta-2 microglobulin.

The MHC monomers can be isolated from appropriate cells or can be recombinantly produced, for example as described by Paul et al, Fundamental Immunology, 2d Ed., W. E. Paul, ed., Ravens Press N.Y. 1989, Chapters 16-18) and readily modified, as described below.

The term “isolated” as applied to MHC monomers herein refers to an MHC glycoprotein heavy chain of MHC class I, which is in other than its native state, for example, not associated with the cell membrane of a cell that normally expresses MHC. This term embraces a full-length subunit chain, as well as a functional fragment of the MHC monomer. A functional fragment is one comprising an antigen binding site and sequences necessary for recognition by the appropriate T cell receptor. It typically comprises at least about 60-80%, typically 90-95% of the sequence of the full-length chain. As described herein, the “isolated” MHC subunit component may be recombinantly produced or solubilized from the appropriate cell source.

It is well known that native forms of “mature” MHC glycoprotein monomers will vary somewhat in length because of deletions, substitutions, and insertions or additions of one or more amino acids in the sequences. Thus, MHC monomers are subject to substantial natural modification, yet are still capable of retaining their functions. Modified protein chains can also be readily designed and manufactured utilizing various recombinant DNA techniques well known to those skilled in the art and described in detail, below. For example, the chains can vary from the naturally occurring sequence at the primary structure level by amino acid substitutions, additions, deletions, and the like. These modifications can be used in a number of combinations to produce the final modified protein chain.

In general, modifications of the genes encoding the MHC monomer may be readily accomplished by a variety of well-known techniques, such as site-directed mutagenesis. The effect of any particular modification can be evaluated by routine screening in a suitable assay for the desired characteristic. For instance, a change in the immunological character of the subunit can be detected by competitive immunoassay with an appropriate antibody. The effect of a modification on the ability of the monomer to activate T cells can be tested using standard in vitro cellular assays or the methods described in the example section, below. Modifications of other properties such as redox or thermal stability, hydrophobicity, susceptibility to proteolysis, or the tendency to aggregate are all assayed according to standard techniques.

Amino acid sequence modification of MHC monomers prepared with various objectives in mind, including increasing the affinity of the subunit for antigenic peptides and/or T cell receptors, facilitating the stability, purification and preparation of the subunits are contemplated to be within the scope of this invention. The monomers may also be modified to modify plasma half-life, improve therapeutic efficacy, or to lessen the severity or occurrence of side effects during therapeutic use of complexes of the present invention. The amino acid sequence modifications of the subunits are usually predetermined variants not found in nature or naturally occurring alleles. The variants typically exhibit the same biological activity (for example, MHC-peptide binding) as the naturally occurring analogue.

Insertional modifications of the present invention are those in which one or more amino acid residues are introduced into a predetermined site in the MHC monomer and which displace the preexisting residues. For instance, insertional modifications can be fusions of heterologous proteins or polypeptides to the amino or carboxyl terminus of the subunits.

Other modifications include fusions of the monomer with a heterologous signal sequence and fusions of the monomer to polypeptides having enhanced plasma half-life (ordinarily>about 20 hours) such as immunoglobulin chains or fragments thereof as is known in the art.

Substitutional modifications are those in which at least one residue has been removed and a different residue inserted in its place. Nonnatural amino acid (i.e., amino acids not normally found in native proteins), as well as isosteric analogs (amino acid or otherwise), are also suitable for use in this invention.

Substantial changes in function or immunological identity are made by selecting substituting residues that differ in their effect on maintaining the structure of the polypeptide backbone (e.g., as a sheet or helical conformation), the charge or hydrophobicity of the molecule at the target site, or the bulk of the side chain. The substitutions which in general are expected to produce the greatest changes in function will be those in which (a) a hydrophilic residue, e.g., serine or threonine, is substituted for (or by) a hydrophobic residue, e.g. leucine, isoleucine, phenylalanine, valine or alanine; (b) a cysteine or proline is substituted for (or by) any other residue; (c) a residue having an electropositive side chain, e.g., lysine, arginine, or histidine, is substituted for (or by) an electronegative residue, e.g., glutamine or aspartine; or (d) a residue having a bulky side chain, e.g., phenylalanine, is substituted for (or by) one not having a side chain, e.g., glycine.

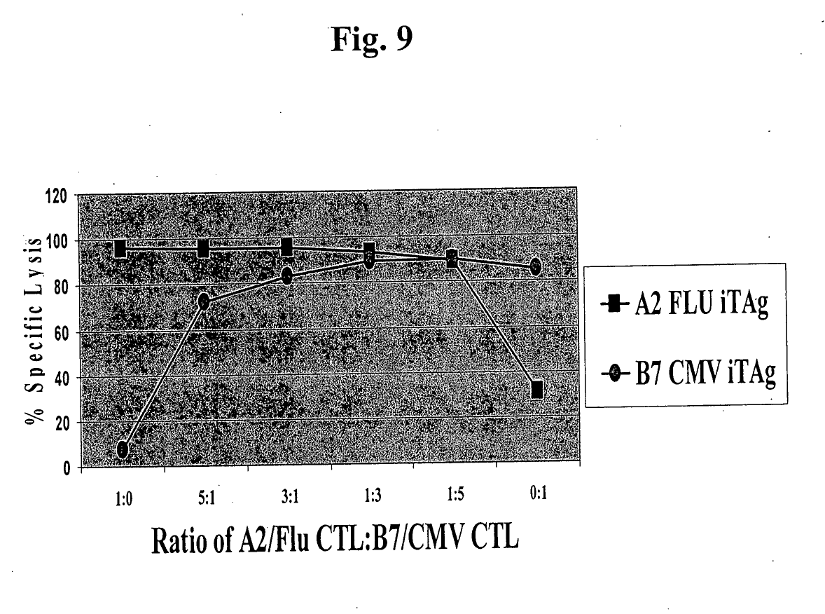

Substitutional modifications of the monomers also include those where functionally homologous (having at least about 70% homology) domains of other proteins are substituted by routine methods for one or more of the MHC subunit domains. Particularly preferred proteins for this purpose are domains from other species, such as murine species as illustrated in FIG. 9 herein.

Another class of modifications is deletional modifications. Deletions are characterized by the removal of one or more amino acid residues from the MHC monomer sequence. Typically, the transmembrane and cytoplasmic domains are deleted. Deletions of cysteine or other labile residues also may be desirable, for example in increasing the oxidative stability of the MHC complex. Deletion or substitutions of potential proteolysis sites, e.g., ArgArg, is accomplished by deleting one of the basic residues or substituting one such residue by a glutaminyl or histidyl residue.

A preferred class of substitutional or deletional modifications comprises those involving the transmembrane region of the subunit. Transmembrane regions of MHC monomers are highly hydrophobic or lipophilic domains that are the proper size to span the lipid bilayer of the cellular membrane. They are believed to anchor the MHC molecule in the cell membrane. Inactivation of the transmembrane domain, typically by deletion or substitution of transmembrane domain hydroxylation residues, will facilitate recovery and formulation by reducing its cellular or membrane lipid affinity and improving its aqueous solubility. Alternatively, the transmembrane and cytoplasmic domains can be deleted to avoid the introduction of potentially immunogenic epitopes. Inactivation of the membrane binding function is accomplished by deletion of sufficient residues to produce a substantially hydrophilic hydropathy profile at this site or by substitution with heterologous residues, which accomplish the same result.

A principal advantage of the transmembrane-inactivated MHC monomer is that it may be secreted into the culture medium of recombinant hosts. This variant is soluble in body fluids such as blood and does not have an appreciable affinity for cell membrane lipids, thus considerably simplifying its recovery from recombinant cell culture. Typically, modified MHC monomers of this invention will not have a functional transmembrane domain and preferably will not have a functional cytoplasmic sequence. Such modified MHC monomers will consist essentially of the effective portion of the extracellular domain of the MHC monomer. In some circumstances, the monomer comprises sequences from the transmembrane region (up to about 10 amino acids), so long as solubility is not significantly affected.

For example, the transmembrane domain may be substituted by any amino acid sequence, e.g., a random or predetermined sequence of about 5 to 50 serine, threonine, lysine, arginine, glutamine, aspartic acid and like hydrophilic residues, which altogether exhibit a hydrophilic hydropathy profile. Like the deletional (truncated) monomer, these monomers are secreted into the culture medium of recombinant hosts.

Glycosylation variants are included within the scope of this invention. They include variants completely lacking in glycosylation (unglycosylated) and variants having at least one less glycosylated site than the native form (deglycosylated) as well as variants in which the glycosylation has been changed. Included are deglycosylated and unglycosylated amino acid sequence variants, deglycosylated and unglycosylated subunits having the native, unmodified amino acid sequence. For example, substitutional or deletional mutagenesis is employed to eliminate the N- or O-linked glycosylation sites of the subunit, e.g., the asparagine residue is deleted or substituted for by another basic residue such as lysine or histidine. Alternatively, flanking residues making up the glycosylation site are substituted or deleted, even though the asparagine residues remain unchanged, in order to prevent glycosylation by eliminating the glycosylation recognition site. Additionally, unglycosylated MHC monomers that have the amino acid sequence of the native monomers are produced in recombinant prokaryotic cell culture because prokaryotes are incapable of introducing glycosylation into polypeptides.

Glycosylation variants are conveniently produced by selecting appropriate host cells or by in vitro methods. Yeasts, for example, introduce glycosylation that varies significantly from that of mammalian systems. Similarly, mammalian cells having a different species (e.g., hamster, murine, insect, porcine, bovine or ovine) or tissue origin (e.g., lung, liver, lymphoid, mesenchymal or epidermal) than the MHC source are routinely screened for the ability to introduce variant glycosylation as characterized for example by elevated levels of mannose or variant ratios of mannose, fucose, sialic acid, and other sugars typically found in mammalian glycoproteins. In vitro processing of the subunit typically is accomplished by enzymatic hydrolysis, e.g., neuraminidase digestion.

MHC glycoproteins suitable for use in the present invention have been isolated from a multiplicity of cells using a variety of techniques including solubilization by treatment with papain, by treatment with 3M KCl, and by treatment with detergent. For example, detergent extraction of Class I protein followed by affinity purification can be used. Dialysis or selective binding beads can then remove detergent. The molecules can be obtained by isolation from any MHC I bearing cell, for example from an individual suffering from a targeted cancer or viral disease.

Isolation of individual heavy chain from the isolated MHC glycoproteins is easily achieved using standard techniques known to those skilled in the art. For example, the heavy chain can be separated using SDS/PAGE and electroelution of the heavy chain from the gel (see, e.g., Domair et al., supra and Hunkapiller, et al., Methods in Enzymol. 91:227-236 (1983). Separate subunits from MHC I molecules are also isolated using SDS/PAGE followed by electroelution as described in Gorga et al. J. Biol. Chem. 262:16087-16094 (1987) and Dornmair et al. Cold Spring Harbor Symp. Quant. Biol. 54:409-416 (1989). Those of skill will recognize that a number of other standard methods of separating molecules can be used, such as ion exchange chromatography, size exclusion chromatography or affinity chromatography.

Alternatively, the amino acid sequences of a number of Class I proteins are known, and the genes have been cloned, therefore, the heavy chain monomers can be expressed using recombinant methods. These techniques allow a number of modifications of the MHC monomers as described above. For instance, recombinant techniques provide methods for carboxy terminal truncation, which deletes the hydrophobic transmembrane domain. The carboxy termini can also be arbitrarily chosen to facilitate the conjugation of ligands or labels, for example, by introducing cysteine and/or lysine residues into the molecule. The synthetic gene will typically include restriction sites to aid insertion into expression vectors and manipulation of the gene sequence. The genes encoding the appropriate monomers are then inserted into expression vectors, expressed in an appropriate host, such as E. coli, yeast, insect, or other suitable cells, and the recombinant proteins are obtained.

As the availability of the gene permits ready manipulation of the sequence, a second generation of construction includes chimeric constructs. The α₁, α₂, α₃, domains of the class I heavy chain are linked typically by the α₃ domain of Class I with beta-2 microglobulin and coexpressed to stabilize the complex. The transmembrane and intracellular domains of the Class I gene can optionally also be included.

Construction of expression vectors and recombinant production from the appropriate DNA sequences are performed by methods known in the art. Standard techniques are used for DNA and RNA isolation, amplification, and cloning. Generally enzymatic reactions involving DNA ligase, DNA polymerase, restriction endonucleases, and the like, are performed according to the manufacturer's specifications. These techniques and various other techniques are generally performed according to Sambrook et al., Molecular Cloning—A Laboratory Manual, Cold Spring Harbor Laboratory, Cold Spring Harbor, N.Y., 1989. The procedures therein are believed to be well known in the art.

Expression can be in procaryotic or eucaryotic systems. Suitable eucaryotic systems include yeast, plant and insect systems, such as the Drosophila expression vectors under an inducible promoter. Procaryotes most frequently are represented by various strains of E. coli. However, other microbial strains may also be used, such as bacilli, for example Bacillus subtilis, various species of Pseudomonas, or other bacterial strains. In such procaryotic systems, plasmid vectors that contain replication sites and control sequences derived from a species compatible with the host are used. For example, E. coli is typically transformed using derivatives of pBR322, a plasmid derived from an E. coli species by Bolivar et al., Gene (1977) 2:95. Commonly used procaryotic control sequences, which are defined herein to include promoters for transcription initiation, optionally with an operator, along with ribosome binding site sequences, including such commonly used promoters as the β-lactamase (penicillinase) and lactose (lac) promoter systems (Change et al., Nature (1977) 198:1056) and the tryptophan (trp) promoter system (Goeddel et al., Nucleic Acids Res. (1980) 8:4057) and the lambda-derived P_{L} promoter and N-gene ribosome binding site (Shimatake et al., Nature (1981) 292:128). Any available promoter system compatible with procaryotes can be used.

The expression systems useful in the eucaryotic hosts comprise promoters derived from appropriate eucaryotic genes. A class of promoters useful in yeast, for example, includes promoters for synthesis of glycolytic enzymes, including those for 3-phosphoglycerate kinase (Hitzeman, et al., J. Biol. Chem. (1980) 255:2073). Other promoters include, for example, those from the enolase gene (Holland, M. J., et al. J. Biol. Chem. (1981) 256:1385) or the Leu2 gene obtained from YEp13 (Broach, J., et al., Gene (1978) 8:121). A Drosophila expression system under an inducible promoter (Invitrogen, San Diego, Calif.) can also be used.

Suitable mammalian promoters include the early and late promoters from SV40 (Fiers, et al., Nature (1978) 273:113) or other viral promoters such as those derived from polyoma, adenovirus II, bovine papilloma virus or avian sarcoma viruses. Suitable viral and mammalian enhancers are cited above.

The expression system is constructed from the foregoing control elements operably linked to the MHC sequences using standard methods, employing standard ligation and restriction techniques, which are well understood in the art. Isolated plasmids, DNA sequences, or synthesized oligonucleotides are cleaved, tailored, and religated in the form desired.

Site-specific DNA cleavage is performed by treatment with the suitable restriction enzyme (or enzymes) under conditions which are generally understood in the art, and the particulars of which are specified by the manufacturer of these commercially available restriction enzymes. In general, about 1 μg of plasmid or DNA sequence is cleaved by one unit of enzyme in about 20 μl of buffer solution; an excess of restriction enzyme may be used to insure complete digestion of the DNA substrate. After each incubation, protein is removed by extraction with phenol/chloroform, and may be followed by ether extraction, and the nucleic acid recovered from aqueous fractions by precipitation with ethanol followed by running over a Sephadex G-50 spin column. If desired, size separation of the cleaved fragments may be performed.

Restriction cleaved fragments may be blunt ended by treating with the large fragment of E. coli DNA polymerase I (Klenow) in the presence of the four deoxynucleotide triphosphates (dNTPs). After treatment with Klenow, the mixture is extracted with phenol/chloroform and ethanol precipitated followed by running over a Sephadex G-50 spin column.

Synthetic oligonucleotides are prepared using commercially available automated oligonucleotide synthesizers. In the proteins of the invention, however, a synthetic gene is conveniently employed. The gene design can include restriction sites that permit easy manipulation of the gene to replace coding sequence portions with these encoding analogs.

Correct ligations for plasmid construction can be confirmed by first transforming E. coli strain MM294 (obtained from E. coli Genetic Stock Center, CGSC #6135), or other suitable host, with the ligation mixture. Successful transformants can be selected by ampicillin, tetracycline or other antibiotic resistance or by using other markers depending on the mode of plasmid construction, as is understood in the art. Plasmids from the transformants are then prepared, optionally following chloramphenicol amplification. The isolated DNA is analyzed by restriction and/or sequenced by the dideoxy method of Sanger, F., et al., Proc. Natl. Acad. Sci. USA (1977) 74:5463 as further described by Messing, et al., Nucleic Acids Res. (1981) 9:309, or by the method of Maxam, et al., Methods in Enzymology (1980) 65:499.

The constructed vector is then transformed into a suitable host for production of the protein. Depending on the host cell used, transformation is done using standard techniques appropriate to such cells. The calcium treatment employing calcium chloride, as described by Cohen, S. N., Proc. Natl. Acad. Sci. USA (1972) 69:2110, or the RbCl method described in Maniatis, et al., Molecular Cloning: A Laboratory Manual (1982) Cold Spring Harbor Press, p. 254 is used for procaryotes or other cells which contain substantial cell wall barriers. For mammalian cells without such cell walls, the calcium phosphate precipitation method of Graham and van der Eb, Virology (1978) 52:546 or electroporation is preferred. Transformations into yeast are carried out according to the method of Van Solingen, P., et al., J. Bacter. (1977) 130:946 and Hsiao, C. L., et al., Proc. Natl. Acad. Sci. USA (1979) 76:3829.

The transformed cells are then cultured under conditions favoring expression of the MHC sequence and the recombinantly produced protein recovered from the culture.

MHC-binding Peptides

It is believed that the presentation of antigen by the MHC glycoprotein on the surface of antigen-presenting cells (APCs) occurs subsequent to the hydrolysis of antigenic proteins into smaller peptide units. The location of these smaller segments within the antigenic protein can be determined empirically. These MHC-binding peptides are thought to be about 8 to about 10, possibly about 8 to about 11, or about 8 to about 12 residues in length, and contain both the agretope (recognized by the MHC molecule) and the epitope (recognized by T cell receptor on the T cell). The epitope is a contiguous or noncontiguous sequence of about 5-6 amino acids that is recognized by the antigen-specific T cell receptor (TCR). The agretope is a continuous or noncontiguous sequence that is responsible for binding of the peptide with the MHC glycoproteins.

Since in the invention compositions and methods the MHC tetramers and MHC multimers are used to bind with or detect CTLs in an antigen-specific manner, the antigenic peptide is selected based on the specificity of the CTLs to be detected, or to which binding is desired. Antigenic peptides that are bound by MHC molecules and presented to T cells are well known in the art and include, for example, a MARTI specific peptide, an HIVgag specific peptide, an HIVpol specific peptide, and the like (see Example 1; see, also, Lang and Bodinier, Transfusion 41:687-690, 2001; Pittet et al., Intl. Immunopharm. 1:12351247,2001; U.S. Pat. No. 6,037,135; Intl. Publ. No. WO 94/20127; Intl. Publ. No. WO 97/34617).

An “MHC monomer” is exemplified herein by major histocompatibility complex (MHC) class I molecules, including class IA molecules and class IB molecules having a MHC-binding peptide bound in the MHC binding pocket. MHC class IA molecules are exemplified by murine H2 molecules, such as H2-D, H2-K and H2-L molecules, and human lymphocyte antigen (HLA) molecules, such as an HLA-A, HLA-B and HLA-C molecules, and MHC class IB molecules are exemplified by HLA-E, HLA-F and HLA-G molecules.

An “MHC multimer” as the term is used herein means a complex of two or more, usually four up to about fifty or more MHC monomers. For example, a yeast cell that recombinantly expresses multiple MHC monomers on its surface or a liposome to which multiple MHC monomers are attached at the surface forms an MHC multimer using the yeast cell or liposome as the multivalent entity that binds the multimer together. More generally, the “multivalent entity” used to bind together an MHC multimer is a molecule, such as streptavidin, with multiple specific binding sites to which an MHC monomer modified with a specific binding site for the multivalent entity will bind.

As used herein, the term “complex”, as distinguished from “bridging complex”, is used broadly to refer to any two molecules, particularly proteins, that specifically associate with each other under physiological conditions. The term “complex” also includes a specific association of two or more molecular complexes. The term “MHC monomer” is used more specifically herein to refer to a complex formed between an MHC class I molecule, θ2-microglobulin, and an MHC-binding peptide, which generally is specifically bound to the peptide binding pocket (cleft) of an MHC class I molecule. An MHC monomer can further contain a peptide sequence engineered into the class I component of the monomer, for example, a signal sequence containing a biotinylation site for the BirA enzyme; and can contain a detectable label. The term “MHC multimer” or “multimeric MHC monomer or modified MHC monomer complex” is used herein to refer to a complex containing two or more MHC monomers, usually bound together via a multivalent entity. An MHC multimer can comprise an MHC dimer, MHC trimer, MHC tetramer, and the like (see, for example, U.S. Pat. No. 5,635,363, which is incorporated herein by reference). The MHC monomers in an MHC multimer can also be linked directly, for example, through a disulfide bond, or indirectly, for example, through a specific binding pair, and also can be associated through a specific interaction between secondary or tertiary structures of the monomers, such as a leucine zipper, which can be engineered, for example, into a MHC class I molecule component of the monomers. MHC tetramers are complexes of four MHC monomers, which are associated with a specific peptide antigen and contain a fluorochrome (U.S. Pat. No. 5,635,363).

MHC class I monomers have been prepared by substituting the transmembrane and cytoplasmic domains of the heavy chain with a peptide sequence that can be biotinylated, and MHC class I tetramers have been formed by contacting such monomers with streptavidin, which can bind four biotin moieties (see, for example, Altman et al., Science 274:94-96, 1996; Ogg and McMichael, Curr. Opin. Immunol. 10:393-396, 1998, each of which is incorporated herein by reference; see, also, U.S. Pat. No. 5,635,363), and are commercially available (Immunomics/Beckman Coulter, Inc.).

MHC tetramers have been prepared using MHC class I molecules, including mutated class IA HLA molecules, including HLA-A*0201, HLA-B*3501, HLA-A*1101, HLA-B*0801, and HLA-B*2705 to minimize binding of the HLA molecules to cell surface CD8 (Ogg and McMichael, supra, 1998). The designation “m” is used to indicate that the class IA molecule is a mutant; for example, HLA-A*0201m is generated from HLA-A*0201 by introducing an A245V substitution (see, for example, Bodinier et al., Nat. Med. 6:707-710, 2000). MHC tetramers containing mutated HLA molecules have a greatly diminished binding to the general population of CD8 cells, but retain peptide-specific binding, thus facilitating accurate discrimination of rare, specific T cells (less than 1% of CD8+; Altman et al., supra, 1996). For example, MHC tetramers composed of four HLA-A*0201 MHC class IA molecules, each bound to a specific peptide and conjugated with phycoerythrin (PE), have been prepared (“i Tag™ MHC Tetramer”); Immunomics/Beckman Coulter, Inc.). The HLA-A0201 allele is found in about 40% to 50% of the global population, and has been modified to minimize CD8 mediated binding (Bodinier et al., Nat. Med. 6:707-710, 2000, which is incorporated herein by reference). These complexes bind to a distinct set of T cell receptors (TCRs) on a subset of CD8+ T cells (McMichael and O'Callaghan, J. Exp. Med. 187:1367-1371, 1998, which is incorporated herein by reference). The i TAg™ MHC Tetramer complexes, for example, recognize human CD8+ T cells that are specific for the particular peptide and HLA molecule in the complex. Since specific binding does not depend on a functional pathway, the population identified by these tetramers includes all specific CD8+ cells, regardless of functional status.

The monomers of an MHC tetramer or other MHC multimer can be operatively linked together, covalently or non-covalently, and directly through a physical association or chemical bond or indirectly through the use of a specific binding pair or by attachment to a multivalent entity through the use of a specific binding pair. Alternatively, the monomers of an MHC multimer can be operatively linked to a multivalent entity containing multiple specific attachment sites for MHC monomers. As used herein, the term “operatively linked” or “operatively associated” means that a first molecule and at least a second molecule are joined together, covalently or non-covalently, such that each molecule substantially maintains its original or natural function. For example, where two or more MHC monomers, each of which can specifically bind a peptide antigen, are operatively linked to form an MHC multimer, each of the two or more MHC monomers in the MHC multimer maintains its ability to specifically bind the peptide antigen. Any means can be used for operatively linking the monomers, provided it does not substantially reduce or inhibit the ability of an MHC multimer to present an antigenic peptide to a T cell. Generally, the MHC monomers are linked together or to a multimeric moiety through the heavy chain component of the monomers. Thus, the monomers can be linked, for example, through an interchain peptide bond formed between reactive side groups of the amino acids comprising the heavy chains, through interchain disulfide bonds formed between cysteine residues in the heavy chains, or through any other type of bond that can generally be formed between the chemical groups represented by the amino acid side chains. A convenient means for operatively linking the monomers of an MHC multimer to a multivalent entity utilizes specific attachment sites that are each part of a binding pair. Where the MHC multimer is formed by attachment of the MHC monomers to a multimeric moiety, the monomers and the multivalent entity each provide one of the specific attachment sites that make up a binding pair.

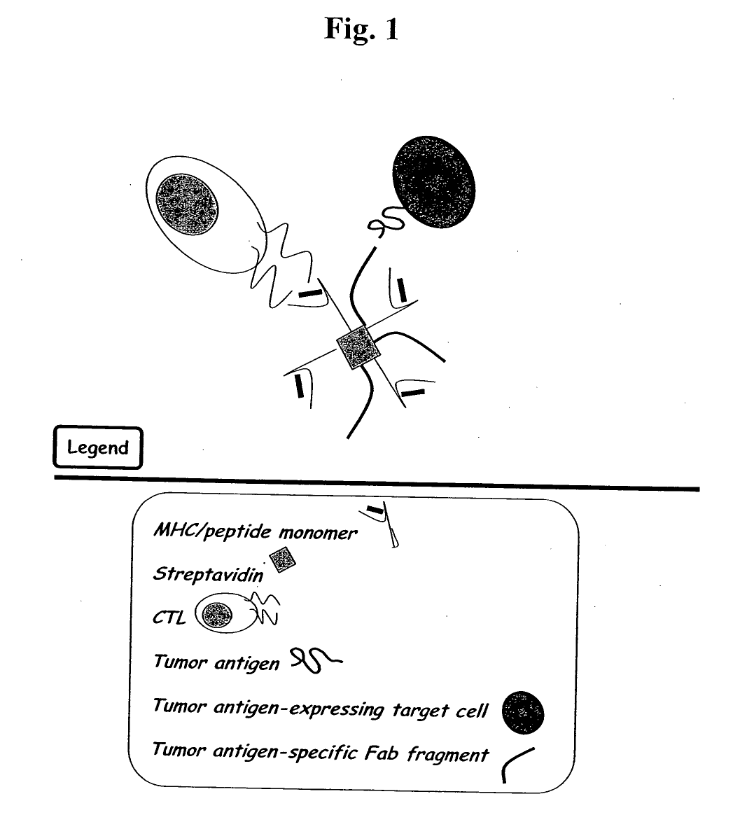

For example, the heavy chains of the monomers can be biotinylated and formed into tetramers by chemical coupling to streptavidin, which naturally has 4 biotin-binding sites (FIG. 1). As used herein, the term “specific binding pair” refers to two molecules that can specifically interact with each other. The two molecules of a specific binding pair can be referred to as “members of a specific binding pair” or as “binding partners.” A specific binding pairs is selected such that the interaction is stable under conditions generally used to perform an immunoassay. Numerous specific binding pairs are well known in the art and include, for example, an antibody that specifically interacts with an epitope and the epitope, for example, an anti-FLAG antibody and a FLAG peptide (Hopp et al., BioTechnology 6:1204 (1988); U.S. Pat. No. 5,011,912); glutathione and glutathione S-transferase (GST); a divalent metal ion such as nickel ion or cobalt ion and a polyhistidine peptide; or the like.

Biotin and streptavidin have been used to prepare MHC tetramers (streptavidin acting as a multivalent entity providing four specific attachment sites for biotin), and biotin and avidin also can be used. These specific binding pairs provide the advantage that a single avidin or streptavidin molecule can bind four biotin moieties, thus providing a convenient means to prepare MHC multimers, such as tetramers. Biotin can be bound chemically to the lysine residues of an MHC heavy chain or can be bound using an enzymatic reaction, wherein the heavy chain is modified to contain a peptide signal sequence comprising a biotinylation site for the enzyme BirA (see Altman et al., supra, 1996; Ogg and McMichael, supra, 1998). Alternatively, biotin can be linked to the θ2-microglobulin, which has fewer lysine residues than an MHC heavy chain, or can be linked to a mutant beta-2 microglobulin, which has been mutagenized to contain only a single accessible lysine residue.

The term “antibody” is used broadly herein to include polyclonal and monoclonal antibodies, as well as antigen binding fragments of such antibodies, such as a Fab, with the proviso that an antibody that binds specifically to a tumor antigen as ligand on a target cell used in the invention constructs and methods and which is chemically attached to streptavidin used as the multivalent entity in a tetramer complex is specifically excluded from the invention.

The term “specifically binds” or “specifically interacts,” when used in reference to an antibody means that an interaction of the antibody and a particular epitope has a dissociation constant of at least about 1×10⁻⁶, generally at least about 1×10⁻⁷, usually at least about 1×10⁻⁸, and particularly at least about 1×10⁻⁹ or 1×10⁻¹⁰ or less. As such, Fab, F(ab′)₂, Fd and Fv fragments of an antibody that retain specific binding activity for a θ2-microglobulin epitope are included within the definition of an antibody. The term “specifically binds” or “specifically interacts” is used similarly herein to refer to the interaction of members of a specific binding pair, as well as to an interaction between θ2-microglobulin and an MHC class I heavy chain.

Depending on the particular method of the invention, antibodies having an Fc region are especially useful in forming the bridging complex of the invention when the target cell of the bridging complex expresses an FcR as the antibody-binding ligand.

In general, the term “antibody” as used herein includes naturally occurring antibodies as well as non-naturally occurring antibodies, including, for example, single chain antibodies, chimeric antibodies, bifunctional or bispecific antibodies and humanized antibodies, as well as antigen-binding fragments thereof. Such non-naturally occurring antibodies can be constructed using solid phase peptide synthesis, can be produced recombinantly or can be obtained, for example, by screening combinatorial libraries consisting of variable heavy chains and variable light chains (see Huse et al., Science 246:1275-1281, 1989). These and other methods of making, for example, chimeric, humanized, CDR-grafted, single chain, and bifunctional or bispecific antibodies are well known to those skilled in the art (Winter and Harris, Immunol. Today 14:243-246, 1993; Ward et al., Nature 341:544-546, 1989; Harlow and Lane, Antibodies: A laboratory manual (Cold Spring Harbor Laboratory Press, 1988); Hilyard et al., Protein Engineering: A practical approach (IRL Press 1992); Borrabeck, Antibody Engineering, 2d ed. (Oxford University Press 1995)).

An antibody having a desired specificity can be obtained using well-known methods. For example, an antibody having substantially the same specific binding activity of C21.48A can be prepared using methods as described by Liabeuf et al. (supra, 1981) or otherwise known in the art (Harlow and Lane, Antibodies: A laboratory manual (Cold Spring Harbor Laboratory Press 1988)). For example, an antibody that specifically binds a specific peptide ligand on the surface of a cell can be obtained using the tumor marker Fc receptor or a peptide portion thereof as an immunogen and removing antibodies that bind with other antigens. A cell surface marker is an antigenic peptide that is suitable for distinguishing a particular type of cell associated with a specific disease state that is present on the cell surface when expressed by the cell. Such antigenic peptides can be identified using crystallographic data or well known protein modeling methods (see, for example, Shields et al., J. Immunol. 160:2297-2307, 1998; Pedersen et al., Eur. J. Immunol. 25:1609, 1995; Evans et al., Proc. Natl. Acad. Sci., USA 79:1994, 1995; Garboczi et al., Proc. Natl. Acad. Sci., USA 89:3429-3433, 1992; Fremont et al., Science 257:919, 1992, each of which is incorporated herein by reference).

Monoclonal antibodies also can be obtained using methods that are well known and routine in the art (Kohler and Milstein, Nature 256:495, 1975; Coligan et al., supra, 1992, sections 2.5.1-2.6.7; Harlow and Lane, supra, 1988). For example, spleen cells from a mouse immunized with a tumor marker or peptide tag, or an epitopic fragment thereof, can be fused to an appropriate myeloma cell line such as SP/02 myeloma cells to produce hybridoma cells. Cloned hybridoma cell lines can be screened using, for example, labeled θ2-microglobulin to identify clones that secrete monoclonal antibodies having the appropriate specificity, and hybridomas expressing antibodies having a desirable specificity and affinity can be isolated and utilized as a continuous source of the antibodies. Polyclonal antibodies similarly can be isolated, for example, from serum of an immunized animal. Such isolated antibodies can be further screened for the inability to specifically bind the cell surface tumor marker an Fc receptor or an expressed peptide tag on a cell surface. Such antibodies, in addition to being useful for performing a method of the invention, also are useful, for example, for preparing standardized kits.

Monoclonal antibodies, for example, can be isolated and purified from hybridoma cultures by a variety of well-established techniques, including, for example, affinity chromatography with Protein-A SEPHAROSE gel, size exclusion chromatography, and ion exchange chromatography (Barnes et al., in Meth. Mol. Biol. 10:79-104 (Humana Press 1992); Coligan et al., supra, 1992, see sections 2.7.1-2.7.12 and sections 2.9.1-2.9.3). Methods of in vitro and in vivo multiplication of monoclonal antibodies are well known. For example, multiplication in vitro can be carried out in suitable culture media such as Dulbecco's Modified Eagle Medium or RPMI 1640 medium, optionally replenished by a mammalian serum such as fetal calf serum or trace elements and growth sustaining supplements such as normal mouse peritoneal exudate cells, spleen cells, bone marrow macrophages. Production in vitro provides relatively pure antibody preparations and allows scale-up to yield large amounts of the desired antibodies. Large-scale hybridoma cultivation can be carried out by homogenous suspension culture in an airlift reactor, in a continuous stirrer reactor, or in immobilized or entrapped cell culture. Multiplication in vivo can be carried out by injecting cell clones into mammals histocompatible with the parent cells, for example, syngeneic mice, to cause growth of antibody-producing tumors. Optionally, the animals can be primed with a hydrocarbon, for example, oil such as pristane (tetramethylpentadecane) prior to injection. After one to three weeks, the desired monoclonal antibody is recovered from the body fluid of the animal.

In certain embodiments, antibodies and antigen binding fragments of antibodies are useful in forming invention bridging complexes, such as those that contain an Fc region and an antigen-binding region, such as a Fab or F(ab′)₂. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly (see, for example, Goldenberg, U.S. Pat. No. 4,036,945 and U.S. Pat. No. 4,331,647; Nisonhoff et al., Arch. Biochem. Biophys. 89:230. 1960; Porter, Biochem. J. 73:119, 1959; Edelman et al., Meth. Enzymol., 1:422 (Academic Press 1967); Coligan et al., supra, 1992, see sections 2.8.1-2.8.10 and 2.10.1-2.10.4).

Furthermore, antibodies or fragments used in the invention bridging complex systems must contain two specific binding sites for operational binding to two different specific binding sites, one located on the surface of the target cell and the other located on the multimeric entity, e.g., on the MHC multimer, incorporated in the bridging complex system. Note that the target cell must not bear a TCR with the same specificity as the CTL in the bridging complex. Thus, in addition to the antigen-binding portion of the antibody, which forms a specific binding pair with an antigen for which it is specific, the antibody has an additional specific binding site. For example, the antibody can be modified to incorporate a biotin to act as a specific binding pair with streptavidin or avidin contained in the MHC multimer. Or the antibody can be modified, as is known in the art and described herein, to contain a polyhisidine tag (for example six hisidine residues) to act as a specific binding pair with a Ni ion. In another example, if the antibody or antigen-binding fragment thereof contains an Fc region, the Fc region can act as a specific attachment site for an Fc receptor, such as may be present as a cell surface ligand on a target cell used in the invention bridging complex systems and methods of use.

In certain embodiments, the invention bridging complex is formed by an antibody with its two specific binding sites forming the “bridge” between the MHC multimer and the target cell. In these embodiments, the two specific binding sites on the antibody are selected to perform this function. As an example, if the multivalent entity used in formation of the MHC multimer has a lipid surface (e.g., a liposome) containing a moiety that chelates nickel, an antibody containing a polyhistidine tag to act as specific binding partner for the chelated nickel on the lipid surface might be selected to have an antigen binding portion that binds specifically to an antigen molecule, such as a tumor marker or recombinant ligand, expressed on the surface of the target cell. Thus binding of the antibody with each of its specific binding partners forms a “bridge” between the MHC multimer and the target cell

In other embodiments, the bridging complex is formed by use of modified tetramers that express a peptide tag that can be recognized by a peptide-specific antibody. In this embodiment, the antibody or antigen-binding fragment is genetically encoded by the hybridoma target cell and is therefore expressed (i.e. “attached”) as an endogenous cell surface protein. Alternatively, MHC monomers can be genetically constructed to contain a peptide moiety that has an antibody recognition site. Using this approach, antibodies specific for this peptide can be used to form a bridge between the multimer in two fashions: attachment by the Fc receptor of the antibody, or use of an antibody-expressing hybridoma target cell. For example, the target cell can be a hybridoma that expresses an antibody on its surface that binds specifically to a histidine tag and the MHC multimer can be designed to contain a polyhistidine tag or to express a polyhistidine tag.

In yet another embodiment, an Fc region of the antibody or antigen-binding fragment can be used to bind specifically to an Fc receptor (expressed either naturally or recombinantly) on the surface of a target cell while the antigen-binding region of the antibody or fragment is selected to bind specifically to a molecule on the surface of the multivalent entity used in formation the MHC multimer. For example, if the multivalent entity used in formation of the MHC multimer is a yeast cell engineered to expresses multiple MHC monomers on its surface, the antibody can be specific for a protein tag also expressed on the surface of the yeast cell.

Methods of cleaving antibodies, such as separation of heavy chains to form monovalent light/heavy chain fragments, further cleavage of fragments, or other enzymatic, chemical, or genetic techniques can also be used, provided the fragments specifically bind to the antigen that is recognized by the intact antibody. For example, Fv fragments comprise an association of variable heavy (V_{H}) chains and variable light (V_{L}) chains, which can be a noncovalent association (Inbar et al., Proc. Natl. Acad. Sci., USA 69:2659, 1972). Alternatively, the variable chains can be linked by an intermolecular disulfide bond or cross-linked by chemicals such as glutaraldehyde (Sandhu, Crit. Rev. Biotechnol. 12:437, 1992).

Antibodies used in the methods of this invention, which are conducted in vitro, can be derived from any species (e.g., goat, murine, rabbit, human, bovine, equine, and the like). Although not a necessity for in vitro uses, humanized monoclonal antibodies also can be used in formation of a bridging complex, a method or kit of the invention if desired. Humanized monoclonal antibodies can be produced, for example, by transferring nucleotide sequences encoding mouse complementarity-determining regions from heavy and light variable chains of the mouse immunoglobulin into a human variable domain, and then substituting human residues in the framework regions of the murine counterparts. Methods for cloning murine immunoglobulin variable domains are known (see, for example, Orlandi et al., Proc. Natl. Acad. Sci., USA 86:3833, 1989), and for producing humanized monoclonal antibodies are well known (see, for example, Jones et al., Nature 321:522, 1986; Riechmann et al., Nature 332:323, 1988; Verhoeyen et al., Science 239:1534, 1988; Carter et al., Proc. Natl. Acad. Sci., USA 89:4285, 1992; Singer et al., J. Immunol. 150:2844, 1993; Sandhu, supra, 1992).

Antibodies useful in a method of the invention also can be derived from human antibody fragments, which can be isolated, for example, from a combinatorial immunoglobulin library (see, for example, Barbas et al., Methods: A Companion to Methods in Immunology 2:119, 1991; Winter et al., Ann. Rev. Immunol. 12:433, 1994). Cloning and expression vectors that are useful for producing a human immunoglobulin phage library are commercially available (Stratagene; La Jolla Calif.). In addition, the antibody can be derived from a human monoclonal antibody, which can be obtained from transgenic mice that have been “engineered” to produce specific human antibodies in response to antigenic challenge (see, for example, by Green et al., Nature Genet. 7:13, 1994; Lonberg et al., Nature 368:856, 1994; and Taylor et al., Int. Immunol. 6:579, 1994; see, also, Abgenix, Inc.; Fremont Calif.).

A method of the invention is performed under any conditions typically used to perform an immunoassay, including a sandwich immunoassay or a competition immunoassay (see Example 2). As such, the reaction can be performed at a temperature of about 4° C. to 37° C., including, for example, at room temperature (about 18° C. to 23° C.), and for a period of time of about 30 minutes to 24 hours, for example, about 1 hour, or overnight (about 12 to 18 hours). The reaction also is performed generally in an aqueous solution, which can contain a buffer such that the pH of the reaction is maintained, if desired, in a relatively narrow range, for example, within about one pH unit of about pH 5, pH 7, or pH 9, and further can contain about a physiological concentration of sodium chloride or other suitable salt.

The invention provides systems, kits, and assays for evaluating putative MHC-binding peptides to determine whether such fragments can be incorporated into a ternary complex with an MHC monomer or modified MHC monomer to activate a specific CTL to lyse a target cell.

Thus, the invention provides systems, kits and screening methods that can be used in screening of candidate peptides for use in diagnostic assays, vaccines, and other treatment modalities. Putative MHC-binding peptides and known MHC binding-peptides for use in the invention methods can be made using any method known in the art, the most convenient being peptide synthesis for fragments of 8 to 12 amino acids in length.

Tetramer Bridging.

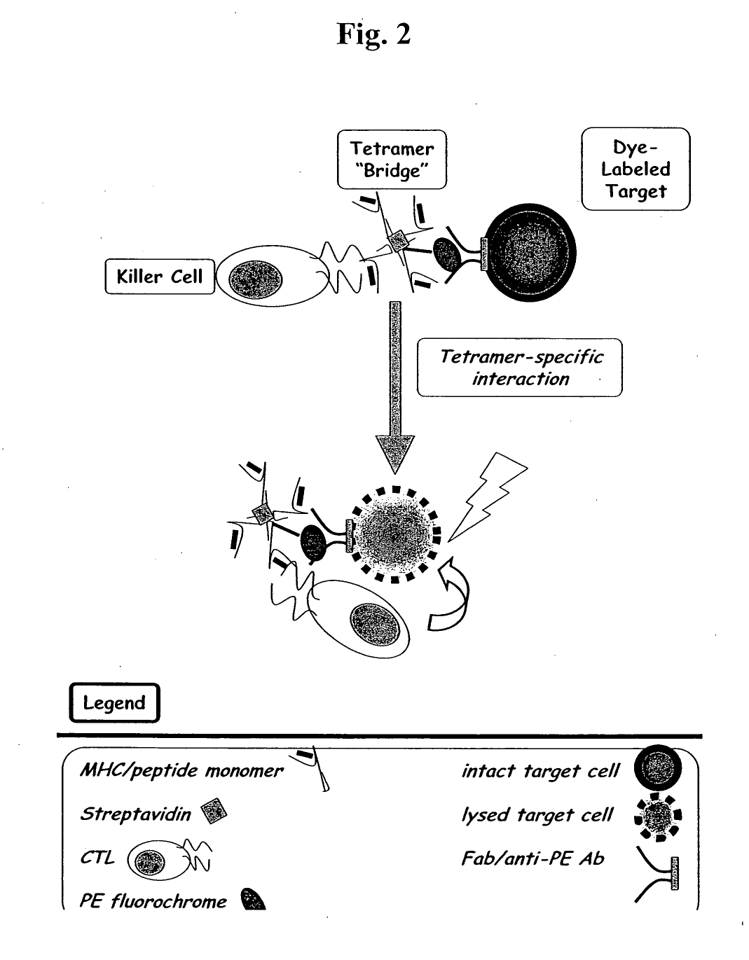

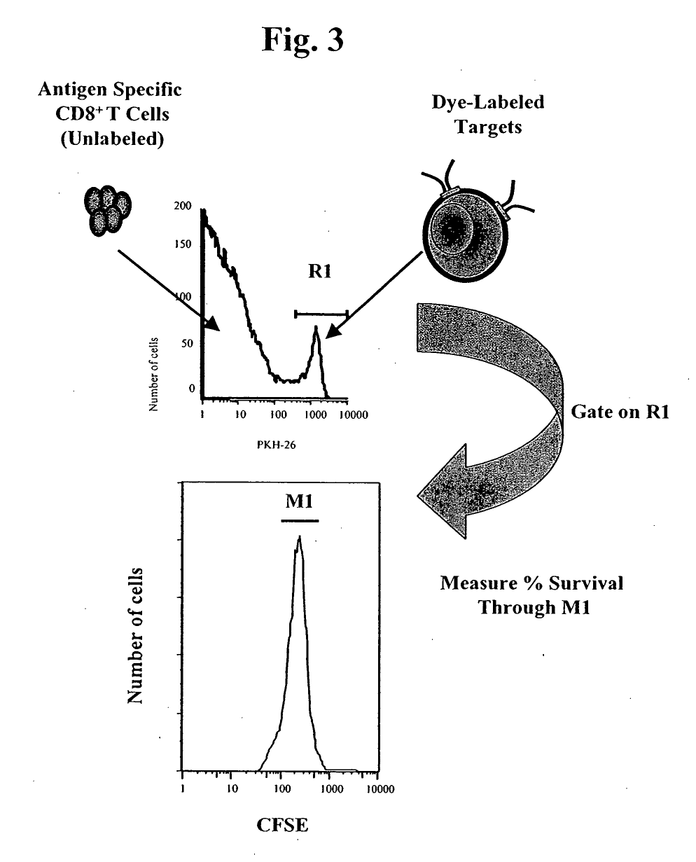

FIG. 2 depicts the concept of tetramer bridging. As shown in FIG. 3, for flow cytometric analysis, target cells are labeled with two fluorometric dyes: CFSE used as the first detectable label and PKH-26 used as the second detectable label. CFSE is an uncharged fluorescein derivative that permeates the cell membrane of target cells and is cleaved by cell enzymes to produce a charged form. The total CFSE signal produced from labeled target cells changes (i.e., decreases) upon lysing of target cells in a peptide-specific manner because CFSE leaks from lysed cells. PKH-26, on the other hand, is a lipophilic dye that labels all target cells uniformly to produce a bright and distinct population easily identified by flow cytometry and does not leak from or become dimmed in lysed target cells. Therefore, the PKH-26 signal remains substantially unchanged upon lysing of target cells. Thus, when membrane damage occurs due to cell lysis, the overall dye fluorescence from the target cells decreases, cells are no longer able to uptake or retain the charged dye, and flow cytometric analysis can be used as illustrated in FIG. 3 to determine the number of target cells that have been lysed.

In the Examples described herein, P815 cells have been used as targets, and these cells express Fc receptor on the cell surface. Anti-PE antibody (Biomeda, Foster City, Calif.) was used to decorate the target cell surface prior to incubation with CTL. Upon incubation with a CTL that has bound tetramer on the cell surface, the TCR/iTAg/Ab/Fc receptor complex, or “bridging complex,” brings the CTL and target into close proximity so as to allow for lysis of the labeled target cell by the CTL to occur in a peptide specific manner.

Kits for performing such methods also are provided. As disclosed herein, the immunoassay methods of the invention are robust, accurate, sensitive, and reproducible.

In one embodiment, the invention provides a bridging complex system useful for detecting or measuring effector function of a MHC monomer containing an MHC-binding peptide for redirecting effector function of a CTL activated by the MHC monomer to a target cell. The invention bridging system is an improvement on MHC tetramer assays because it can be used to determine not only antigen-specific binding of monomers to TCRs but also antigen-specific effector function of activated CTLs for a target cell. Moreover, the invention bridging system can be used to redirect effector function of such a monomer-activated CTL to a target cell that does not bear a surface peptide for which the TCR of the CTL in the bridging complex is peptide specific.

The invention bridging system is a complex comprising a cytotoxic T cell (CTL) having a T cell receptor (TCR) specific for an antigenic peptide bound in the binding pocket of an MHC monomer or modified MHC monomer; a multimeric MHC monomer or modified MHC monomer complex having at least one antibody attachment site; and an antibody having a first attachment site that forms a binding pair with the attachment site (e.g., specific for a ligand) on target cell and a second attachment site that forms a binding pair with the antibody attachment site on the multimeric MHC monomer complex. Binding of antibody to the MHC monomer complex via respective binding pairs and to the target cell via the attachment site (or ligand) forms a bridging complex such that the CTL is brought into proximity sufficient for a peptide-specific or monomer-activated CTL to lyse the target cell. For the CTL used in the invention bridging complex system to have effector activity so as to lyse a target cell, the CTL must be monomer-activated (i.e., the CTL must be CD8+ and have effector function, rather than being either naïve or anergic, neither of which exhibit effector functions). For example, in clinical settings, a patient sample containing CTL activated in vivo by a disease-associated antigen can be assayed for effector function using an invention method wherein such a bridging complex forms.

In an embodiment, illustrated in FIG. 1, the multivalent entity in the bridging system is streptavidin or avidin and an antigen-specific CTL is associated with a tumor antigen-expressing target cell by a Fab′ antibody fragment specific for the cell surface tumor antigen by chemical coupling the antibody to the streptavidin. At least one and up to four biotinylated MHC/peptide complexes (i.e. MHC monomers) can be bound together by streptavidin, which naturally has 4 specific attachment sites for biotin-binding sites. The invention bridging complex can optionally also comprise a target cell that has a cell surface attachment site that forms a binding pair with the first attachment site on the antibody.

Mixing the MHC tetramers with peripheral blood lymphocytes or whole blood, and using flow cytometry as a detection system can obtain a count of all antigen-specific CTLs in the sample. As such, the MHC tetramers or multimers allow for the measurement of a cellular response against a specific peptide.

In this embodiment, the invention bridging system is useful for detecting effector function of the CTL for a target cell bearing a specific cell surface attachment site to which the antibody attaches to form a binding pair. Thus, for example, if the antibody is selected to have antigenic specificity for a tumor marker found on the surface of a tumor cell and the antibody is chemically attached to the multivalent entity (the chemical bond providing the binding pair between the two in this case), the invention system can be used, for example, to determine whether a patient sample contains tumor cells expressing the tumor marker by detecting lysing of the target cell by CTLs in the patient sample.

In another embodiment, the target cell with surface antigen can be known, and the invention bridging system can be used to determine whether avidity between a TCR and an MHC monomer presented on the multivalent entity is sufficient to activate a T-cell in an antigen-specific manner.

In one embodiment, the invention bridging complex system comprises streptavidin as the multivalent entity and up to four of the MHC monomers or modified MHC monomers are biotinylated so as to form a binding pair with the streptavidin. An antigen-specific antibody can be chemically coupled to the strepavidin.

In another embodiment, the multivalent entity can be a lipid surface, such as the surface of a liposome containing multiple attachment sites for the monomer and antibody. For example, the multivalent entity can be a liposome containing a lipid modified to bind to a histidine tag and at least one MHC monomer or modified MHC monomer and the antibody or antibody fragment, each having a carboxy terminal histidine tag, can be bound to the surface of the liposome via the histidine tag. For example, lipids containing Ni-iminodiacetic acid (Ni-IDA) or Ni-nitriloacetic acid (Ni-NTA) are binding partners for polyhistidine. An example of a lipid modified to bind to a histidine tag is 1,2-dioleoyl-sn-glycero-3-[N-95 amino-1-carboxypentyl) iminodiacetic acid) succinyl] with covalently attached nickel-chelating group, N″,N″-bis[carboxymethyl]-L-lysine (nitriloacetic acid) (DOGS-Ni-NTA).

In another embodiment, the multivalent entity can be a yeast cell that expresses at least one, and preferably a plurality of the MHC monomers or modified MHC monomers, on the surface of the cell. An antibody can be attached to the yeast cell surface or one or more of the antibodies can be genetically expressed on (i.e., “attached”) the surface of the yeast cell. Alternatively still, the multivalent entity can be a hybridoma and the MHC monomers or modified MHC monomers can be expressed on the surface of the hybridoma. In yet another embodiment, the target cell in the bridging complex can be a hybridoma that expresses on its cell surface a peptide for which the antibody is specific.

The term “target cell(s)” as used herein means any cell, procaryotic or eukaryotic, e.g., mammalian, bacterial, yeast, or insect, that does not bear on its surface a peptide for which the CTL in the bridging complex has peptide-specific effector function, but does bear a surface ligand to serve as an attachment site for an antibody. Thus, the target cell can be a tumor cell that bears a cell surface marker (ligand) that identifies the cell as associated with a particular disease. Alternatively, the target cell can be one that, when transfected with a heterologous nucleotide sequence that encodes a protein ligand or tag, will express the ligand on the surface of the target cell, for example to provide a specific antigen binding site for an antibody. In another example, the cell can be one that is transformed or naturally expresses an Fc receptor on the cell surface to serve as an attachment site for the Fc region of an antibody. In another embodiment, the target cell can be a hybridoma or phage that expresses an antibody or antigen-binding antibody fragment on its surface. For example, the target cell can be one that can be transformed to express an FcR. In this embodiment, an antibody having an Fc region and the FcR on the target cell form a specific binding pair and an antigen for which the antibody is specific is located on the multimeric entity that binds together the multimer such that immunological binding of the antibody to the antigen and the FcR forms the second specific binding pair necessary for formation of the invention bridging complex. In this embodiment, the same target cell-ligand (FcR)-antibody combination can be used to test innumerable different combinations of antigenic peptide-CTL combinations.

The bridging complex formed in practice of the invention methods is labeled with at least one detectable label that is useful for distinguishing whether a bridging complex has been formed, for example by distinguishing target cells contained in a bridging complex (e.g., lysed target cells) from other target cells used in the assay. A fluorescent molecule, a radionuclide, a luminescent molecule, a chemiluminescent molecule, an enzyme, or a peptide such as a polyhistidine tag, a myc epitope, or a FLAG™ epitope can be incorporated into the MHC monomer or multimer used in the invention methods. For example, MHC tetramers comprising a fluorescent phycoerythrin label are commercially available (Immunomics). Radionuclides, particularly ⁵¹Cr are commonly used in assays to detect lytic activity.

Alternatively, in certain embodiments designed for high throughput screening, the target cells used in the invention compositions, methods and kits can be labeled with at least one detectable label that is useful for distinguishing whether the target cell is intact or has been lysed by a CTL when incorporated into an invention bridging complex. Any type of label known in the art that can be used to make this distinction can be used. However, lipophilic dyes, particular lipophilic fluorescent dyes that are substantially non-toxic to living cells and which leak from lysed cells or labels that change color when the target cell is lysed are preferred. In this embodiment, the first detectable label is preferably selected to be a fluorescent dye that is lipophilic and hence can be used to stain cells, but becomes charged when exposed to cell contents, such as esterase found in some amount in most mammalian cells, and thus leaks from lysed cells. A second criterion for selection of a first detectable label is that the emission wavelengths of the first and second detectable labels are sufficiently distinguishable for cell sorting, either manually or by fluorescence activated cell sorting (FACS) assay. For example, when fluorescein is protected as the diacetate, it becomes nonpolar enough to passively cross the plasma membrane of living cells. Due to esterase activity in the cytosol, the intracellular dye is cleaved back to fluorescein, giving bright green fluorescence. Depending on the polarity (charge) on the fluorescein derivative, it can remain trapped in the cytosol for long periods. CFSE is presently preferred for use as the first detectable label in invention methods. CFSE is also known as CSFE, CMDA-AM and CFDA in the scientific literature. There is some academic debate as to whether CFSE actually leaks from lysed cells or is “dimmed” following a pH change within the target cell.

To detect the number of target cells in a sample containing multiple target cells, the target cells can be additionally labeled with a second detectable label with a signal that does not change upon lysing of the target cells. Non-limiting examples of lipophilic fluorescent dyes that readily stain target cells include lipophilic carbocyanine or aminostyryl. Lipophilic carbocyanines DiI (DiICl₁₈, DiO (DiOC₁₈, DiD (DiIC₁₈ and DiR DiIC₁₈) are weakly fluorescent in water but highly fluorescent and quite photostable when incorporated into cell membranes. These fluorescent dyes have extremely high extinction coefficients (>125,000 cm⁻¹M⁻¹ at their longest-wavelength absorption maximum) though modest quantum yields, and short excited-state lifetimes (˜1 nanosecond) in lipid environments. Once applied to cells, the dyes diffuse laterally within the plasma membrane, resulting in staining of the entire cell. Transfer of these probes between intact membranes is usually negligible. Another example of a lipophilic dye that can be used as the second detectable label for target cells in an invention bridging complex, method or kit is a membrane intercalating dye, such as PKH-2 and PKH-26.

The use of MHC tetramers and other MHC multimers described herein to analyze T cell specificity provides significant advantages over previously used T cell assays. For example, the invention methods are quantitative, do not require the use of radioactive dyes, and are readily adapted to high throughput assay formats. In addition, the invention methods can be performed quickly and, therefore, can be used to examine fresh blood or tissue samples. Where the MHC multimer complex includes a fluorescent label, a cell population including T cells can be further stained with one or more other fluorescently labeled molecules, for example, fluorescently labeled molecules specific for other cell surface molecules, and analyzed using flow cytometry, thus allowing additional characterization of the responding cells. In this case, the additional fluorescent label is selected to fluoresce at a wavelength that is readily distinguishable from the label(s) used to label the bridging complex or stain the target cells. Furthermore, the invention methods are not toxic to the labeled cells and, therefore, cells incorporated into invention bridging complexes can be sorted into uniform populations by flow cytometry and examined by additional assays to confirm their functional ability, for example, the ability to proliferate in response to antigen.

The following examples are intended to illustrate but not limit the invention.

### EXAMPLE 1

Materials and Methods

Reagents. Peptides used for in vitro stimulation of PBMCs (New England Peptide, Inc., Fitchburg, Mass.) throughout these examples were greater than 90% pure, as determined by mass spectrometry and reverse-phase HPLC analysis. Phycoerythrin (PE)-coupled iTAg® tetramers were commercially available (Beckman Coulter, Inc. Immunomics Operations, San Diego, Calif.).

Cell Lines. CD8+ CTL clone HA2FLU.3 and HA2FLU.5, specific for amino acids 58-66 of influenza A matrix peptide (GILGFVFTL) (SEQ ID NO:1) in the context of HLA-A*0201 (Bednarek), and CTL clone B7 CMV.16, specific for amino acids 417-426 of CMV pp65 peptide (TPRVTGGGAM) (SEQ ID NO:2) in the context of HLA-B7 (Wills, M. R., et al., J Virol 1996 November; 70(11):7569-79) were generated as described below.

### EXAMPLE 2

Generation of CD8+ CTLs. PBMCs obtained from normal, healthy volunteers were isolated by centrifugation over Histopaque® 1077 medium (Sigma Diagnostics, St. Louis, Mo.). IM-9 cells and P815 cells were obtained from ATCC and passaged weekly in RPMI-FC(RPMI supplemented with 2 mM L-Glutamine, 1M HEPES, 1 mM Sodium Pyruvate, 0.1 mM non-essential amino acids (all from Invitrogen Life Technologies, Carlsbad, Calif.) and 10% final concentration of Fetal Clone (HyClone Laboratories, Logan, Utah)). For antigen presenting cells (APCs), ten million cells were resuspended in RPMI-AB (RPMI supplemented with 5×10⁻⁵ M 2-mercaptoethanol (Sigma, St. Louis, Mo.), and the supplements listed for RPMI-FC, except that human serum (10% final concentration, Valley Biomedical, Winchester, Va.) was used instead of Fetal Clone), formalin-fixed staphylococcus aureus Cowan (final concentration of 0.0017%, Sigma Diagnostics), and 500 ng IL-4 (BD Pharmingen, San Diego, Calif.). Cells were seeded at 4×10⁶ cells/ml in a 24-well plate and incubated at 37° C., in 5% CO₂ for two days. For CD8⁺ lymphocytes, an additional 10 million cells were depleted of CD4⁺ cells using DYNABEADS® beads (Dynal Biotech, Lake Success, N.Y.) according to the manufacturer's instructions.

Depleted cells were resuspended at a concentration of 2×10⁶/ml in RPMI-AB supplemented with 100 U/ml IL-2 (R&D). One ml per well was added into wells of 24 well plates and incubated at 37° C. in 5% CO₂ for two days. On the day of CTL stimulation, APCs were harvested and resuspended at 6×10⁶/ml in RPMI (no additives). Peptide was added to APCs at a concentration of 40 mg/ml and incubated at 37° C. for 45 minutes. An equal volume of 2×RPMI-AB (RPMI-AB containing 20% human serum and 2× of the supplements) was then added, and APCs were added to wells of 96 well round-bottom plates in 100 ml aliquots. CD4 minus depleted PBMC were harvested, counted, and resuspended at 3×10⁵ cells/ml in RPMI-AB. One hundred microliters of cells were added to each well containing APC.

After 2 days, recombinant IL-2 was added to plates to give a final concentration of 100 U/ml. Expanding wells were screened 12-18 days later, and positive wells were subcloned by limiting dilution. Briefly, graded numbers of lymphocytes from positive wells were co-cultured in Terisaki plates with 2×10⁵ irradiated PBMC and PHA at a final concentration of 5 ug/ml. Two weeks later, positive wells were expanded and screened for tetramer binding. CTL clones were routinely expanded by culturing with PHA and 5×1 05 irradiated IM-9 cells in 24 well plates.

### EXAMPLE 3

Tetramer Staining. CTLs were harvested, counted and resuspended in PBS 0.1% BSA at various concentrations in a final volume of 100 μl. Ten microliters of specific or irrelevant tetramer (iTAg® reagents, Beckman Coulter Immunomics, San Diego, Calif.) was added, and samples were incubated for 20-30 minutes at 4° C. Samples to be used for phenotypic analysis were co-stained with CD8-FITC during this incubation step. Samples were resuspended in flow cytometry buffer (azide and BSA) prior to analysis on a Becton Dickinson FASCcaliber® or a Beckman Coulter EPICS XL® flow cytometer.

### EXAMPLE 4

Method for Analysis of Cytoeffector Function. CTL were assayed 16-21 days after stimulation. Target cells were prepared by staining cells with PKH-26 dye (Sigma) and carboxy fluorescein diacetate, succinimidyl ester (CFSE) (Molecular Probes, Eugene, Oreg.) as described by Sheehy et al. (J. Immunol. Methods (2001) 249:99-110). Briefly, P815 cells were washed in RPMI, no additives and resuspended in 1 ml of Diluent C. PKH-26 in a volume of 1 ml Diluent C (Sigma Chemicals) was added to cells to give a final concentration of 2.5×1⁻⁷ M and incubated for 5 minutes. The reaction was stopped by addition of an equal volume of fetal bovine serum (FBS). Cells were washed and resuspended in 1 ml PBS, and CFSE was added to give a final concentration of 2.5×10⁻⁷ M. An equal volume of FBS was immediately added to the cells to stop the reaction. Cells were washed and resuspended in PBS 0.1% BSA. One hundred microliters of targets were incubated on ice for 30 minutes with 100 ng of anti-phycoerythrin Ab or isotype control. Targets were then washed, counted, and resuspended in RPMI-AB at a concentration of 1×10⁵/ml. One-hundred microliters containing 1×10⁴ targets were added to each well of a 96 well round-bottom plate that contained titrated amounts of tetramer-labeled CTLs to give the appropriate Effector:Target ratio (E:T). Plates were incubated at 37° C. in 5% CO₂ for 4 hours. Samples were then harvested, washed, and fixed with 1% paraformaldehyde prior to flow cytometry analysis. For each data point, 3-4 replicates were performed, and combined prior to analysis.

In order to detect effector function, assays were performed using FATAL (fluorometric assessment of T lymphocyte antigen specific lysis) analysis, as described by Sheehy et al. Both CFSE and PKH-26 were excited at 488 nm. CFSE fluorescence was detected at 505-555 nm with an FL1 detector of a flow cytometer using a 530/25 filter. PKH-26 fluorescence was detected at 545-625 nm with an FL2 detector of the flow cytometer using a 585/40 filter. During flow cytometric analysis, target cells are differentiated from CTL in each sample by virtue of PKH-26 dye (FIG. 3). A gate is drawn on PKH-26-positive cells in order to quantitate the amount of CFSE remaining in the target cells, as measured by marking CFSE^{hi} target cells, and determining the amount of remaining CFSE, as compared to target cells incubated in the absence of CTL (see Materials and Methods).

Detection of Bridging-Mediated Lysis by CTL Clones.

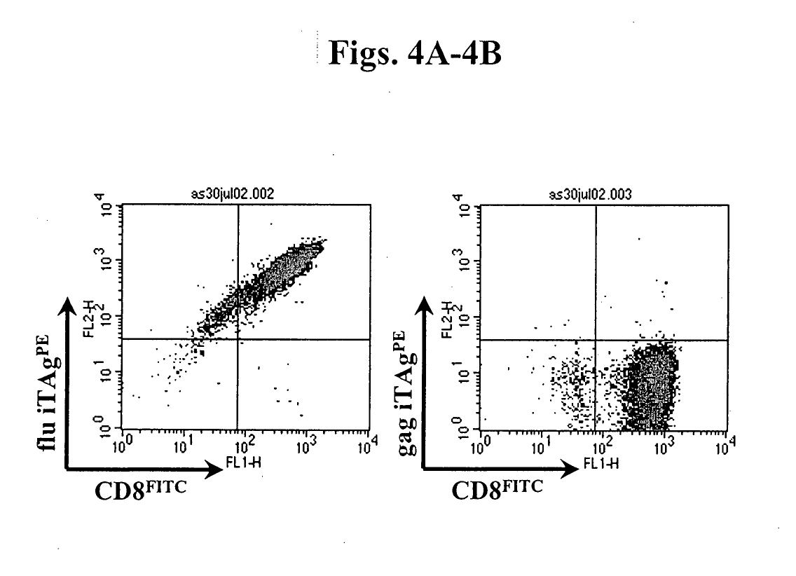

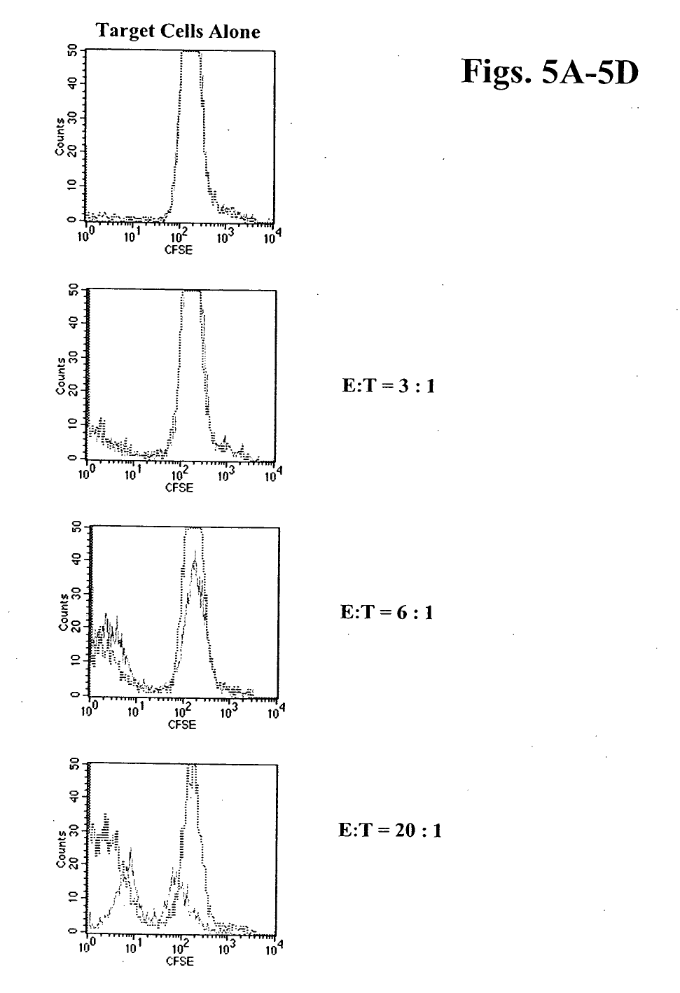

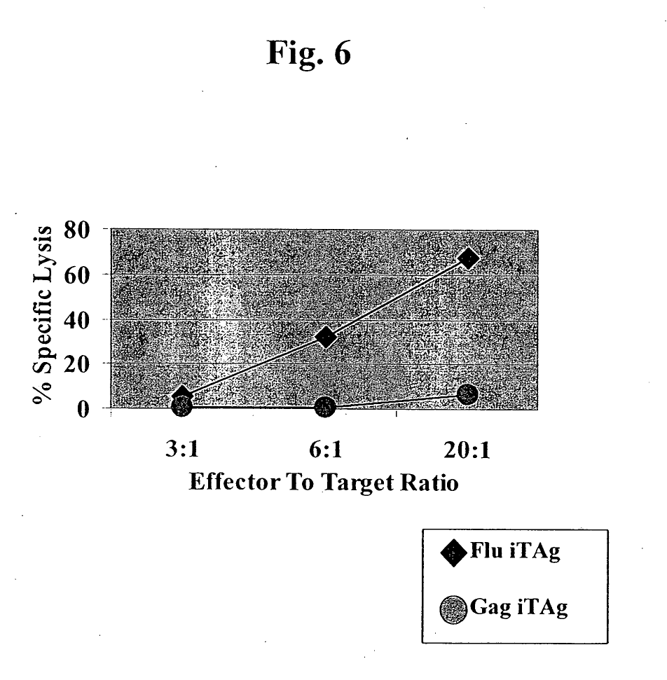

HA2FLU.3 is a CTL clone generated to recognize influenza matrix peptide in the context of HLA-A*0201. These CTL were incubated with A2/Flu tetramer or an MHC-matched tetramer with an irrelevant peptide (A2/gag tetramer, FIG. 4). The results showed that this CTL clone is CD8⁺ and specifically binds A2/Flu tetramer with high avidity. To determine the validity of the bridging concept, HA2FLU.3 cultures were labeled with A2/Flu or A2/CMV tetramer, and this time samples were then co-incubated with anti-PE-decorated, dye-labeled P815 targets at 3 different Effector:Target ratios. Flow cytometric analysis of these samples revealed a significant reduction in the CFSE signal from the target cells at the highest Effector:Target ratio, and the signal gradually increased as the Effector:Target ratio decreased (FIG. 5). Importantly, this inverse correlation of CFSE signal and Effector:Target ratio was only observed when target cells were incubated with CTL that were incubated with the specific tetramer. CTL incubated with irrelevant tetramer did not cause a decrease in the CFSE signal. When lytic activity was calculated, the data showed that only A2/Flu tetramer-stained CTL were able to cause lysis of targets through bridging, at levels significantly higher than those observed with CTL reacted with the irrelevant A2/CMV tetramer (FIG. 6). Together, these experiments showed that a CTL clone restricted to the HLA-A*0201 allele was able to mediate lysis of target cells by virtue of their ability to specifically bind MHC tetramer.

B. Bridging-Mediated Lysis by Mixed CTL.

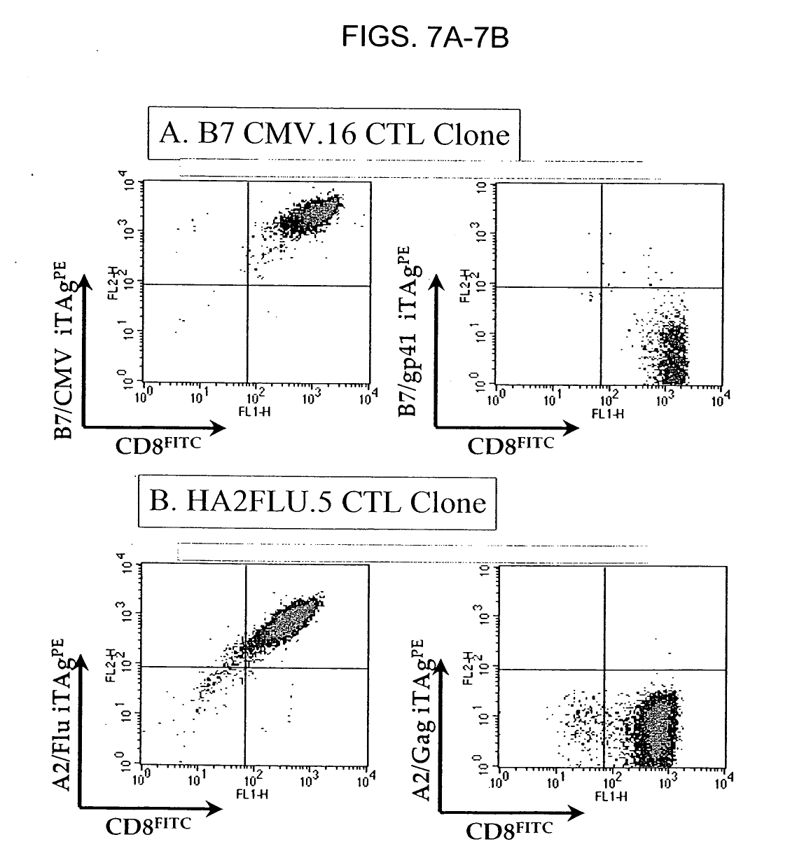

The next experiment was designed to answer two questions: 1) whether CTL of a different allele could be induced to lyse targets through bridging, and 2) whether the bridging assay could detect effector function in a mixed CTL population of samples stained reciprocally with specific tetramers. CTL clones B7 CMV. 16 and HA2FLU.5 were prepared as described above in Example 2. CTL clone B7 CMV. 16 is a CTL clone that is restricted to CMV pp65 peptide in the context of HLA-B*0702, and binds specific tetramer with high avidity, as compared to CTL incubated with an MHC-matched irrelevant tetramer B7/gp41 (FIG. 7A). HA2FLU.5 is a CTL clone that behaves similarly to sister clone HA2FLU.3 used in earlier experiments, and binds relevant tetramer with high avidity (FIG. 7B). These two CTL clones were mixed in ratios beginning at 1:0 HA2FLU.5:B7CMV. 16, and ending with the opposite ratio (0:1). Samples were mixed and separated into two groups. One group was stained with A2/Flu tetramer, and the other group with the reciprocal B7/CMV tetramer.

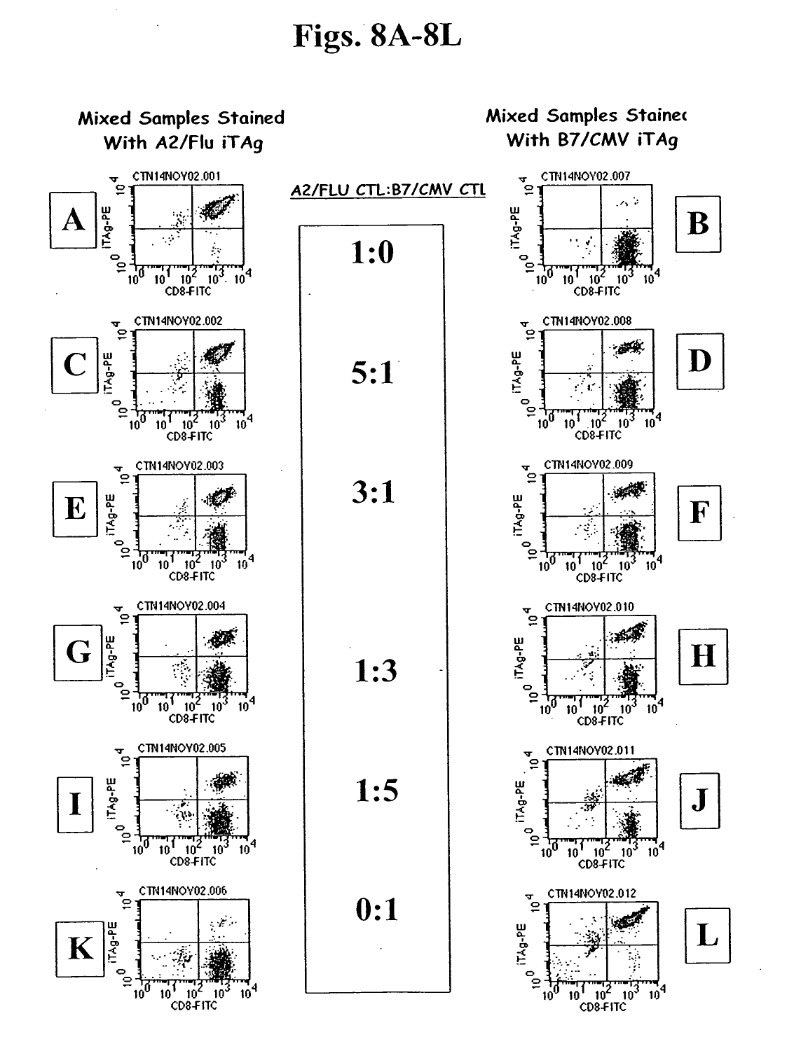

The results of this mixed population-staining experiment (FIG. 8) showed that CTLs were present at different ratios in each sample, and there was little cross-reactivity of CTL with the reciprocal tetramer. Each sample was also incubated with P815 targets decorated with anti-PE Ab, and the beginning E:T of CTL samples not mixed with the other CTL was 60:1, which was significantly higher than the E:T that was used in the earlier bridging experiments. The results of this experiment showed that most of the samples containing A2/Flu lysed targets at high levels, with little titration, probably due to the initially high E:T ratio (FIG. 9). Interestingly, the sample containing only B7/CMV CTL (0:1) could not bind A2/Flu tetramer, yet was able to lyse targets at approximately 30% specific lysis. The group of CTL mixtures incubated with B7 tetramer showed better titration, and the background was significantly lower. Samples containing only HA2FLU.5 (E:T ratio=1:0) were unable to lyse target cells in the presence of B7/CMV tetramer, and background levels were comparable to those observed with the sister CTL clone HA2FLU.3.

The high E:T ratio used in this example did not allow for optimal titration of effector function. In addition, the cross reactivity of the B7/CMV CTL clone was probably directed to the P815 target cells, which, in addition to FcR, express murine class I MHC, with which human CTL may exhibit cross-reactivity. The results may be more clear-cut using a different FcR+ target, or a different CTL clone that has low cross-reactivity, as do the A2/Flu CTL clones.

Although the invention has been described with reference to the above examples, it will be understood that modifications and variations are encompassed within the spirit and scope of the invention. Accordingly, the invention is limited only by the following claims.

1. An assay for identifying a peptide of a known antigen that induces peptide-restricted effector function in a CTL, said assay comprising:
a) co-incubating under suitable conditions so as to allow interaction between:
1) a target cell with a surface ligand and which is tagged with a first detectable label with a signal that is substantially changed in lysed target cells as compared to unlysed target cells;
2) a multimeric MHC monomer of modified MHC monomer complex with bound test MHC-binding peptide, wherein the complex further comprises an antibody-specific binding site;
3) an antibody that binds to the ligand on the target cells and to the antibody-specific binding site; and
4) a peptide-restricted CTL,
wherein the interaction results in formation of a bridging complex that brings the peptide-restricted CTL into cell-lysing proximity of the target cells; and
b) detecting a change in signal produced by target cells in the bridging complex as compared with signal produced by uncomplexed target cells, wherein the change identifies the peptide in the MHC class I monomer as inducing peptide-restricted effector function in the CTL.

2. The method of claim 1, wherein the ligand is an Fc receptor expressed on the surface of the target cell so that binding of the antibody to the antibody binding site and to the Fc receptor through Fc/FcR interaction forms the bridging complex.

3. The method of claim 1, wherein the target cell is a cell associated with a disease and the ligand is a surface marker of the disease.

4. The method of claim 3, wherein the target cell is a tumor cell and the ligand is a cell surface tumor marker.

5. The method of claim 1, wherein the detectable label is a fluorescent dye or a radiolabel.

6. The method of claim 1, wherein the change is a substantial decrease in signal from the first detectable label.

7. The method of claim 1, wherein the multimeric MHC complex comprises streptavidin with two to four biotinylated MHC monomers bound thereto, the antibody binding site is PE attached to the streptavidin, and the ligand is an anti-PE Fab, wherein the bridging complex forms by specific binding of the Fab to the PE and chemical binding of the PE to the streptavidin.

8. The method of claim 7, wherein four biotinylated monomers are bound to the streptavidin.

9. The method of claim 1, wherein the detecting comprises flow cytometric analysis for determining the amount of the target cells with substantially decreased signal intensity, thereby detecting the amount of the CTLs with the effector function.

10. The method of claim 9, wherein the first detectable label is carboxy fluorescein diacetate, succinimidyl ester (CFSE).

11. The method of claim 9, wherein the target cells are also labeled with a second detectable label with a signal that does not change upon lysing of the target cells and the flow cytometric analysis comprises comparing the amount of the first and second labels in the target cells to determine the amount of target cells that retain the first detectable label, thereby determining the amount of the CTLs with the effector function.

12. The method of claim 11, wherein the mutimeric MHC complex comprises a multivalent entity with specific attachment sites for a plurality of the monomers.

13. The method of claim 11, wherein the multivalent entity is a lipid surface with a plurality of the specific attachment sites.

14. The method of claim 11, wherein the multivalent entity is a yeast cell having surface expression of the monomers.

15-50. (canceled)


SEQUENCE LISTING (2 sequences)

SEQ ID NO: 1 - protein, 9 aa, Artificial sequence
  Synthetic construct
GILGFVFTL

SEQ ID NO: 2 - protein, 10 aa, Artificial sequence
  Synthetic construct
TPRVTGGGAM